(19) United States
(12) Patent Application Publication
(10) Pub. No.: US 2026/0207538 A1
(43) Pub. Date: Jul. 23, 2026

(54) COMBINATIONS FOR LIVER DISEASE

(71) Applicant: Gilead Sciences, Inc., Foster City, CA (US)

(72) Inventors: Yun-Jung Choi, Fremont, CA (US); Jeffrey D. Johnson, Greeley, CO (US); Andrew N. Billin, San Mateo, CA (US); Jun Xu, Lexington, MA (US)

(21) Appl. No.: 19/423,508

(22) Filed: Dec. 17, 2025

Related U.S. Application Data
(60) Provisional application No. 63/737,340, filed on Dec. 20, 2024.

(51) Int. Cl. A61K 31/192; A61K 31/235; A61K 31/415; A61K 31/42; A61K 31/4439; A61K 31/454; A61K 31/46; A61K 31/496; A61K 31/575; A61P 1/16

(52) U.S. Cl. CPC A61K 31/192; A61K 31/235; A61K 31/415; A61K 31/42; A61K 31/4439; A61K 31/454; A61K 31/46; A61K 31/496; A61K 31/575; A61P 1/16

(57) ABSTRACT

The present disclosure relates to a method of preventing and/or treating liver disease comprising administering an FXR agonist in combination with seladelpar or a pharmaceutically acceptable salt thereof to a patient in need thereof.

### CROSS REFERENCE TO RELATED APPLICATIONS

This application claims the benefit under 35 U.S.C. 119(e) of U.S. Provisional Application No. 63/737,340, filed on Dec. 20, 2024, which is hereby incorporated by reference in its entirety.

### SEQUENCE LISTING

The instant application contains a Sequence Listing which has been submitted electronically in .XML format and is hereby incorporated by reference in its entirety. Said .XML copy, created on Dec. 11, 2025, is named 403379.xml and is 6,358 bytes in size.

### BACKGROUND

Liver disease is a leading cause of death worldwide. Primary biliary cholangitis (PBC), a chronic liver disease, is an autoimmune condition of the liver marked by the slow progressive destruction of the small bile ducts of the liver, with the intralobular ducts affected early in the condition. When these ducts are damaged, bile builds up in the liver (cholestasis) and over time damages the tissue, which can lead to scarring, fibrosis, and cirrhosis.

There is no cure for PBC, and liver transplantation often becomes necessary; but medication such as ursodeoxycholic acid (UDCA, ursodiol) to reduce cholestasis and improve liver function may slow the progression to allow a normal lifespan and quality of life. UDCA is approved in the United States to treat PBC, but about 40% of patients are reported to have an inadequate response to UDCA and about 5% are reported to be intolerant to UDCA treatment.

Recently, seladelpar lysine, a ppar delta agonist, was conditionally approved in the United States and elsewhere to treat PBC, in combination with ursodeoxycholic acid (UDCA) in adults who have had an inadequate response to UDCA, or as monotherapy in patients unable to tolerate UDCA.

The Farnesoid X Receptor (FXR), also often referred to as NR1H4 (nuclear receptor subfamily 1, group H, member 4) when referring to the human receptor, is a nuclear hormone receptor. FXR has been associated with multiple biological functions. FXR is primarily expressed in the liver and throughout the entire gastrointestinal tract, but is also found in the kidney, adrenal glands, and ovary. FXR is associated with controlling intracellular gene expression, and may be involved in paracrine and endocrine signaling. In the intestine and liver, FXR functions as a regulator of bile acid homeostasis and hepatic lipogenesis. FXR has also been associated with Kupffer cells and liver sinusoidal endothelial cells of the liver, wherein it is believed to have functions related to inflammation, fibrosis, and portal hypertension.

A number of FXR agonists are known and are being investigated in connection with a number of physiological conditions, including liver diseases. FXR agonists can have benefits in steatosis, lobular inflammation, hepatocellular ballooning, and fibrosis.

However, FXR agonists, such as obeticholic acid (OCA), have also been associated with adverse effects, especially pruritus. It has been reported that FXR agonists cause pruritus or worsen pre-existing pruritus caused by underlying condition. However, solutions to the adverse effect of pruritus have not been fully developed.

There remains a need for safe and effective treatments for liver disease and/or adverse effects of liver disease, such as pruritus.

### SUMMARY

According to one aspect, the present disclosure provides a method of treating and/or preventing a liver disease in a patient in need thereof. The method comprises administering a FXR agonist or a pharmaceutically acceptable salt thereof and a therapeutically effective amount of seladelpar or a pharmaceutically acceptable salt thereof to the patient.

In some embodiments, the FXR agonist is obeticholic acid (OCA), GW4064, ASC-42, PX-104, TERN 101, cilofexor, GS-8670, tropifexor, vonafexor, EDP-305, MET-409, or a pharmaceutically acceptable salt thereof. In some embodiments, the FXR agonist is

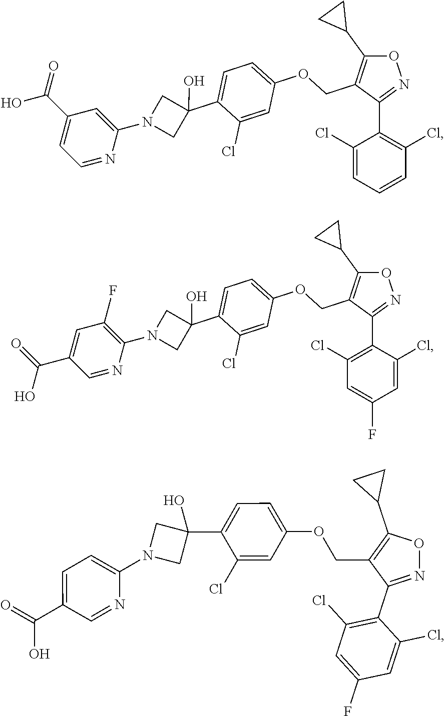

or a pharmaceutically acceptable salt thereof. In some embodiments, the patient is administered seladelpar lysine. In some embodiments, the patient is administered seladelpar L-lysine. In some embodiments, the patient is administered seladelpar L-lysine dihydrate. In some embodiments, the therapeutically effective amount of seladelpar or the pharmaceutically acceptable salt thereof is equivalent to about 10 mg/day of seladelpar. In some embodiments, the liver disease is metabolic dysfunction-associated steatohepatitis (MASH), formally non-alcoholic steatohepatitis (NASH), metabolic dysfunction-associated steatotic liver disease (MASLD), primary biliary cholangitis (PBC), or primary sclerosing cholangitis (PSC). In some embodiments, the patient has been previously administered an FXR agonist and the patient was associated with pruritus. In some embodiments, the pruritus is FXR-induced. In some embodiments, the pruritus is due to the patient being administered an FXR agonist. In some embodiments, the FXR agonist and seladelpar are administered together. In some embodiments, the FXR agonist and seladelpar are administered separately.

In another aspect, the present disclosure provides a method of treating pruritus, such as Farnesoid X Receptor (FXR)-induced pruritus, comprising administering to a patient in need thereof a therapeutically effective amount of seladelpar or a pharmaceutically acceptable salt thereof.

In some embodiments, the patient is suffering from: metabolic dysfunction-associated steatohepatitis (MASH), formally non-alcoholic steatohepatitis (NASH), Primary sclerosing cholangitis (PSC), primary biliary cirrhosis (PBC), autoimmune hepatitis, alcoholic steatohepatitis (ASH), liver fibrosis, liver cirrhosis, liver steatosis (fatty liver disease), liver ischemia, Type I Diabetes, Type II Diabetes, metabolic dysfunction-associated steatotic liver disease (MASLD), Barrett's esophagus, and progressive familiar intrahepatic cholestasis (PFIC). In some embodiments, the patient is suffering from a liver disease. In some embodiments, the patient is suffering from metabolic dysfunction-associated steatotic liver disease (MASLD), primary biliary cholangitis (PBC), or primary sclerosing cholangitis (PSC).

In some embodiments, a patient is suffering from pruritus. In some embodiments, the patient is suffering from FXR-induced pruritus due to the patient being administered an FXR agonist or due to a bile acid. In some embodiments, the FXR agonist is obeticholic acid (OCA), GW4064, ASC-42, PX-104, TERN 101, cilofexor, GS-8670, tropifexor, vonafexor, EDP-305, MET-409, or a pharmaceutically acceptable salt thereof. In some embodiments, the FXR agonist is:

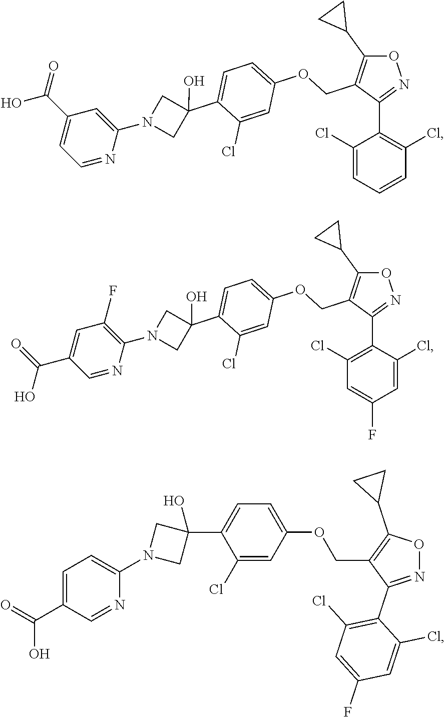

or a pharmaceutically acceptable salt thereof. In some embodiments, the FXR-induced pruritus is associated with elevated serum IL-31 levels due to increased serum bile acid levels.

In another aspect, the present disclosure provides a composition comprising a therapeutically effective amount of an FXR agonist and a therapeutically effective amount of seladelpar or a pharmaceutically acceptable salt thereof.

In some embodiments, the FXR agonist is obeticholic acid (OCA), GW4064, ASC-42, PX-104, TERN 101, cilofexor, GS-8670, tropifexor, vonafexor, EDP-305, MET-409, or a pharmaceutically acceptable salt thereof. In some embodiments, the FXR agonist is:

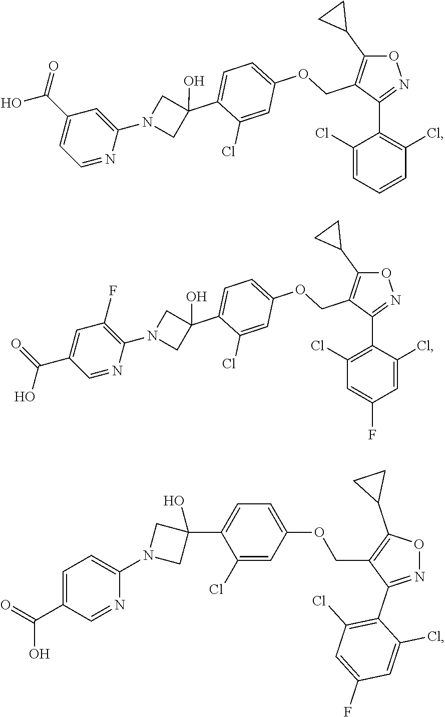

or a pharmaceutically acceptable salt thereof. In some embodiments, the composition comprises seladelpar lysine. In some embodiments, the composition comprises seladelpar L-lysine. In some embodiment, the composition comprises seladelpar L-lysine dihydrate.

### BRIEF DESCRIPTION OF THE DRAWINGS

FIG. 1 is a graph showing levels of individual serum bile acids in patients with PBC compared to healthy volunteers.

FIG. 2 is a graph showing correlations between serum conjugated bile acids in patients with PBC.

FIG. 3 is a graph showing IL-31 protein levels of the primary human hepatocytes treated with FXR agonists and bile acids with or without seladelpar treatment.

FIG. 4 is a graph showing activation of human FXR by FXR agonists and bile acids evaluated in a human FXR reporter gene assay.

FIG. 5 depicts graphs showing IL-31 protein level in primary human hepatocytes treated for 72 hours with different concentrations of CDCA, OCA, or GW4064.

FIG. 6 depicts graphs showing IL-31 protein and IL-31 mRNA level in primary human hepatocytes treated for 72 hours with different concentrations of CDCA with or without DY268 and/or seladelpar.

FIG. 7 illustrates alignment of the genomic sequence of the human IL-31 gene with FXR ChIP-Seq data (GME: SRX530186.bw) and JASPAR-predicted FXR:RXRA binding sites.

FIG. 8 is a graph showing reporter gene activity of IL-31 Exon 3 IR1 sites driven by different concentrations of FXR agonists (CDCA, OCA, and GW4064) in Huh7 cells.

FIG. 9 a graph showing reporter gene activity driven by FXR agonists in Huh7 cells mediated by IL-31 Exon 3 IR1 sites and mutants thereof.

FIG. 10 depicts graphs illustrating Binding of FXR to the IR1 sites, BSEP and IL-31, in the human IL-31 gene.

It will be recognized that some or all of the figures are schematic representations for purpose of illustration.

### DETAILED DESCRIPTION

### Definitions

The following description sets forth exemplary embodiments of the present technology. It should be recognized, however, that such description is not intended as a limitation on the scope of the present disclosure but is instead provided as a description of exemplary embodiments.

As used in the present specification, the following words, phrases, and symbols are generally intended to have the meanings as set forth below, except to the extent that the context in which they are used indicates otherwise.

A dash (“—”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. Also, to the term “about X” includes description of “X.” Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

The terms “optional” or “optionally” means that the subsequently described event or circumstance may or may not occur, and that the description includes instances where said event or circumstance occurs and instances in which it does not. Also, the term “optionally substituted” refers to any one or more hydrogen atoms on the designated atom or group may or may not be replaced by a moiety other than hydrogen.

The term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile, e.g., for injectables. The term “pharmaceutically acceptable salt” of a given compound refers to salts that retain the biological effectiveness and properties of the given compound, and which are not biologically or otherwise undesirable. Pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines. Specific examples of suitable amines include, by way of example only, isopropyl amine, trimethyl amine, diethyl amine, tri(iso-propyl) amine, tri(n-propyl) amine, ethanolamine, diethanolamine, 2-dimethylamino ethanol, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like. Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

As used herein, the term “hydrate” means a complex formed by combination of water molecules with molecules or ions of the solute, and “monohydrate” includes one water molecule, and “dihydrate” incudes two water molecules.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

As used herein, a “FXR agonist” refers to any agent that is capable of binding and activating farnesoid X receptor (FXR) which may be referred to as bile acid receptor (BAR) or NR1H4 (nuclear receptor subfamily 1, group H, member 4) receptor. FXR agonist may act as agonists or partial agonists of FXR. The agent may be a chemical compound or biological molecule (e.g., a protein or antibody).

As used herein, the term “Farnesoid X Receptor (FXR)-induced pruritis” or “FXR-induced pruritis” refers to pruritis brought about by FXR activation. In some embodiments, the FXR induced pruritis is due to the patient being administered an FXR agonist. In some embodiments, the FXR-induced pruritis is associated with elevated serum IL-31 levels due to increased serum bile acid levels.

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Subject” refers to an animal, such as a mammal (including a human), that has been or will be the object of treatment, observation, or experiment. The methods described herein may be useful in human therapy and/or veterinary applications. In some embodiments, the subject is a mammal. In one embodiment, the subject is a human.

“Monitoring” or “monitor” generally refers to the overseeing, supervision, regulation, watching, tracking, or surveillance of an activity, an occurrence, a change, or a condition. For example, the term “monitoring the patient for a symptom” refers to tracking the occurrence of a symptom in a patient. Similarly, the “monitoring,” when used in connection with patient compliance, either individually, or in a clinical trial, refers to the tracking or confirming that the patient is actually taking the compound being tested as prescribed. The monitoring can be performed, for example, by following biomarkers, communicating with the subject or patient, or visual observation. Monitoring may be done in any degree or any frequency which is considered suitable by a clinician or administrator. For example, a patient may be monitored for a certain biomarker very closely and/or very frequently, for example by measuring the biomarker every week, every day, every hour, or real time. On the other hand, a patient may be monitored less often or more passively. For example, a patient may be monitored by asking the patient about occurrence of certain symptom during a monthly, or a bi-monthly follow up meeting, or by instructing the patient to contact and report certain symptom if it occurs. “Monitoring” can also include testing or checking (verbally or non-verbally) for a certain condition, symptom, or biomarker.

The term “therapeutically effective amount” or “effective amount” of a compound described herein or a pharmaceutically acceptable salt, tautomer, stereoisomer, mixture of stereoisomers, prodrug, or deuterated analog thereof means an amount sufficient to effect treatment when administered to a subject, to provide a therapeutic benefit such as amelioration of symptoms or slowing of disease progression. For example, a therapeutically effective amount may be an amount sufficient to decrease a symptom of a disease or condition of a liver disease, such as primary biliary cholangitis (PBC). The therapeutically effective amount may vary depending on the subject, and disease or condition being treated, the weight and age of the subject, the severity of the disease or condition, and the manner of administering, which can readily be determined by one or ordinary skill in the art.

### Methods of Treating

The disclosure provides methods of treating liver disease by providing a combination of seladelpar, or a pharmaceutically acceptable salt thereof, and an FXR agonist, or a pharmaceutically acceptable salt thereof. In some embodiments, methods of treating liver disease comprise administering seladelpar lysine and cilofexor, or a pharmaceutically acceptable salt thereof. In some embodiments, methods of treating liver disease comprise administering seladelpar lysine and GS-8670, or a pharmaceutically acceptable salt thereof.

In some embodiments, the disclosure provides methods of improving cholestasis in a patient in need thereof, by administering a combination of seladelpar, or a pharmaceutically acceptable salt thereof, and an FXR agonist, or a pharmaceutically acceptable salt thereof.

In some embodiments, the disclosure provides methods of reducing bile acids, in a patient in need thereof, by administering a combination of seladelpar, or a pharmaceutically acceptable salt thereof, and an FXR agonist, or a pharmaceutically acceptable salt thereof.

In some embodiments, methods of the disclosure prevent liver fibrosis, cirrhosis, and liver failure. In some embodiments, methods of the disclosure prevent end-stage liver disease.

In some embodiments, treatment with seladelpar, or a pharmaceutically acceptable salt thereof, and an FXR agonist, or a pharmaceutically acceptable salt thereof, provides an improved hepatoprotective benefit to a patient relative to administration with a single therapeutic agent for treatment of a liver disease.

As described herein, both FXR agonists and seladelpar independently may reduce bile acid levels and have potential to benefit patients in liver disease.

However, FXR agonism in the liver can increase pruritus due to FXR mediated transcriptional activation of IL-31, potentially resulting in elevated IL-31 levels and pruritus.

Seladelpar has been shown to improve markers of cholestasis, such as alkaline phosphatase (ALP), gamma-glutamyl transferase (GGT), and total bilirubin (TB) in patients with PBC, and can provide anti-cholestatic, anti-inflammatory, and anti-pruritic effects. It has been reported that seladelpar is a selective ppar delta agonist that suppresses bile acid synthesis by reducing hepatocyte CYP7A via the fibroblast growth factor 21 (FGF 21) signaling pathway.

Seladelpar can negatively regulate IL-31 even in the continuous presence of FXR activation. Accordingly, the combination of a FXR agonist and seladelpar is contemplated to be used for the treatment of FXR mediated conditions and/or cholestasis to reduce the risk of pruritus. Further, other than the reduced risk of pruritus, seladelpar is reported to have antisteatotic effect and improve liver injury, therefore useful for the treatment of liver diseases. Seladelpar has been also approved for the treatment of primary biliary cholangitis (PBC).

Accordingly, the combination of an FXR agonist and seladelpar is contemplated to be effective in treating cholestatic, or FXR-mediated liver diseases. In some embodiments, the combination may further provide reduced risk of pruritus when compared to FXR monotherapy. In some embodiments, the combination may reduce pruritus in a patient suffering from cholestatic disease. In some embodiments, the combination may provide greater hepatoprotective benefits relative to treatment with a single therapeutic agent for treatment of liver disease.

In some embodiments, a method of treating a liver disease is provided, the method comprising administering seladelpar, or a pharmaceutically acceptable salt thereof, and an FXR agonist, or a pharmaceutically acceptable salt thereof. In some embodiments, a method of treating PBC is provided, the method comprising administering seladelpar, or a pharmaceutically acceptable salt thereof, and an FXR agonist, or a pharmaceutically acceptable salt thereof. In some embodiments, the method comprises co-administering seladelpar, or a pharmaceutically acceptable salt thereof, and an FXR agonist, or a pharmaceutically acceptable salt thereof. In some embodiments, the method includes administering a fixed dose combination of seladelpar, or a pharmaceutically acceptable salt thereof, and an FXR agonist, or a pharmaceutically acceptable salt thereof.

In some embodiments, administering a combination of seladelpar, or a pharmaceutically acceptable salt thereof, and an FXR agonist, or a pharmaceutically acceptable salt thereof, to a patient reduces one or more of cholestatic injury, inflammation, or liver fibrosis. In some embodiments, a method of reducing cholestatic injury in a patient comprises administering an FXR agonist and seladelpar, or a pharmaceutically acceptable salt thereof. In some embodiments, a method of reducing inflammation in a patient comprises administering an FXR agonist and seladelpar, or a pharmaceutically acceptable salt thereof. In some embodiments, a method of reducing liver fibrosis in a patient comprises administering an FXR agonist and seladelpar, or a pharmaceutically acceptable salt thereof.

In some embodiments, a method of treating a liver disease is provided, wherein seladelpar, or a pharmaceutically acceptable salt thereof, and an FXR agonist, or a pharmaceutically acceptable salt thereof, is provided to a patient in combination with UDCA. In some embodiments, the method comprises administering UDCA and a fixed dose combination of seladelpar, or a pharmaceutically acceptable salt thereof, and an FXR agonist, or a pharmaceutically acceptable salt thereof.

### FXR Mediated Conditions

Disclosed herein is a method of treating and/or preventing a FXR mediated condition in a patient in need thereof, comprising administering to the patient a therapeutically effective amount of a FXR agonist in combination with a therapeutically effective amount of seladelpar or a pharmaceutically acceptable salt thereof.

Compounds described herein or combinations thereof may be useful for treating or preventing an FXR mediated condition.

FXR regulates a complex pattern of response genes in the liver and in the gastrointestinal tract. The gene products have impact on diverse physiological processes. For example, FXR represses the induction of Cyp7A1 via the upregulation of mRNA encoding SHP, a further nuclear receptor that is dominant repressive over LRH-1. Since FXR binds primary bile acids, the end products of this pathway, this can be regarded as an example of feedback inhibition on the gene expression level.

FXR ligands induce bile flow and change bile acid composition towards more hydrophilic composition. With the development of the first synthetic FXR ligand GW4064 as a tool compound and of the semi-synthetic artificial bile acid ligand 6-alpha-ethyl-CDCA, the effects of superstimulation of FXR by potent agonists could be analyzed. It was shown that both ligands induce bile flow in bile duct ligated animals. Moreover, in addition to choleretic effects, hepatoprotective effects could also be demonstrated. These hepatoprotective effects included anti-fibrotic effects resulting from the repression of Tissue Inhibitors of Matrix-Metalloproteinases TIMP-1 and 2, the induction of collagen-deposit resolving Matrix-Metalloproteinase 2 in hepatic stellate cells, and the subsequent reduction of alpha-collagen mRNA and Transforming growth factor beta (TGF-beta) mRNA, both of which are pro-fibrotic factors.

Furthermore, anti-cholestatic activity was demonstrated in bile-duct ligated animal models as well as in animal models of estrogen-induced cholestasis. Genetic studies demonstrate that in hereditary forms of cholestasis (Progressive Familiar Intrahepatic Cholestasis=PFIC, Type I-IV) either nuclear localization of FXR itself is reduced as a consequence of a mutation in the FIC1 gene (in PFIC Type I, also called Byler's Disease) (F. Chen et al., Gastroenterology 2004, 126, 756; L. Alvarez et al., Hum. Mol. Genet. 2004, 13, 2451) or levels of the FXR target gene encoding MDR-3 phospholipid export pump are reduced (in PFIC Type III). There is a growing body of evidence that FXR binding compounds can demonstrate substantial clinical utility in the therapeutic regimen of chronic cholestatic conditions such as Primary Biliary Cirrhosis (PBC) or Primary Sclerosing Cholangitis (PSC).

FXR agonists can be useful to prevent cholesterol gallstone formation or to prevent re-formation of gallstones after surgical removal or shockwave lithotripsy. For example, using the synthetic FXR tool compound GW4064 it could be demonstrated that activation of FXR leads to an improvement of the Cholesterol Saturation Index (CSI) and directly to an abolishment of gallstone formation in C57L gallstone susceptible mice whereas drug treatment in FXR knockout mice shows no effect on gallstone formation.

Thus, in one embodiment of the disclosure, an FXR mediated condition is an obstructive or chronic inflammatory disorder that arises out of improper bile composition such as cholelithiasis also known as cholesterol gallstones.

FXR agonists can be useful in protecting the intestine from neoplastic transformation and from the development of polyps and their transition into adenocarcinoma in the gut. Absence of FXR leads to a high increase in the formation of Hepatocellular Carcinoma (HCC), the most prominent form of liver cancer. Whereas a functional FXR prevents the formation of colon adenocarcinoma and hepatocellular carcinoma, FXR activation induces liver regeneration after hepatectomy.

The combined hepatoprotective, anti-neoplastic and liver regenerative effects associated with FXR activation can be therapeutically exploited for the use of FXR agonists in the treatment of severe liver diseases.

In one embodiment, the FXR mediated condition is a liver disease, such as HCC, stimulation of liver regrowth and amelioration of side effects associated with major liver resection, liver cirrhosis independent of the etiology and prevention or treatment of liver ischemia in the course of liver transplantation or major liver surgery.

Moreover, FXR can be a key regulator of serum triglycerides. Activation of FXR by synthetic agonists can leads to significant reduction of serum triglycerides, mainly in the form of reduced VLDL, but also to reduced total serum cholesterol. Lowering of serum triglycerides is not a standalone effect. Treatment of db/db or ob/ob mice with synthetic FXR agonist GW4064 resulted in marked and combined reduction of serum triglycerides, total cholesterol, free fatty acids, ketone bodies such as 3-OH Butyrate. Moreover, FXR activation engages with the intracellular insulin signaling pathway in hepatocytes, resulting in reduced output of glucose from liver gluconeogenesis but concomitant increase in liver glycogen. Insulin sensitivity as well as glucose tolerance were positively impacted by FXR treatment. An effect on reduction of body weight was also recently observed in mice overfed with a high lipid diet. This weight loss effect might result from FXR's induction of FGF-19, a fibroblast growth factor that is known to lead to weight loss and athletic phenotype. The effect of FXR agonist on reduction of body weight has been demonstrated.

Accordingly, FXR agonists can be exploited in different therapeutic ways: FXR binding compounds are thought to be good candidates for the treatment of Type II Diabetes because of their insulin sensitization, glycogenogenic, and lipid lowering effects.

In some embodiments, FXR mediated condition is Type II Diabetes which can be overcome by FXR-mediated upregulation of systemic insulin sensitivity and intracellular insulin signaling in liver, increased peripheral glucose uptake and metabolization, increased glycogen storage in liver, decreased output of glucose into serum from liver-borne gluconeogenesis.

In some embodiments, the FXR mediated condition is chronic intrahepatic, such as PBC, PSC, progressive familiar cholestasis (PFIC), alcohol-induced cirrhosis and associated cholestasis, and some forms of extrahepatic cholestatic conditions, or liver fibrosis.

In some embodiments, the FXR mediated condition is a gastrointestinal condition with a reduced uptake of dietary fat and fat-soluble dietary vitamins which can be overcome by increased intestinal levels of bile acids and phospholipids.

In some embodiments, the FXR mediated condition is a lipid and lipoprotein disorder such as hypercholesterolemia, hypertriglyceridemia, and atherosclerosis as a clinically manifest condition which can be ameliorated by FXR's beneficial effect on lowering total plasma cholesterol, lowering serum triglycerides, increasing conversion of liver cholesterol into bile acids and increased clearance and metabolic conversion of VLDL and other lipoproteins in the liver.

In some embodiments, the FXR mediated condition is a disease where the combined lipid lowering, anti-cholestatic and anti-fibrotic effects of FXR-targeted medicaments can be exploited for the treatment of liver steatosis and associated syndromes such as metabolic dysfunction-associated steatohepatitis (MASH), or for the treatment of cholestatic and fibrotic effects that are associated with alcohol-induced cirrhosis, or with viral-borne forms of hepatitis.

In some embodiments, the FXR mediated condition is obesity and associated disorders such as metabolic syndrome (combined conditions of dyslipidemias, diabetes and abnormally high body-mass index) which can be overcome by FXR-mediated lowering of serum triglycerides, blood glucose and increased insulin sensitivity and FXR-mediated weight loss.

In some embodiments, the FXR mediated condition is a clinical complication of Type I and Type II Diabetes. Examples of such complications include Diabetic Nephropathy, Diabetic Retinopathy, Diabetic Neuropathies, or Peripheral Arterial Occlusive Disease (PAOD). Other clinical complications of diabetes are also encompassed by the present disclosure.

In some embodiments, the FXR mediated condition is a condition and disease which result from chronic fatty and fibrotic degeneration of organs due to enforced lipid and specifically triglyceride accumulation and subsequent activation of profibrotic pathways. Such conditions and diseases encompass MASH and chronic cholestatic conditions in the liver, Glomerulosclerosis and Diabetic Nephropathy in the kidney, Macular Degeneration and Diabetic Retinopathy in the eye and neurodegenerative diseases, such as Alzheimer's Disease in the brain, or Diabetic Neuropathies in the peripheral nervous system.

In some embodiments, the FXR mediated condition is congenital hepatic fibrosis.

In some embodiments for the methods described herein, the FXR mediated condition is selected from metabolic dysfunction-associated steatohepatitis (MASH), formally non-alcoholic steatohepatitis (NASH), Primary sclerosing cholangitis (PSC), primary biliary cirrhosis (PBC), autoimmune hepatitis, alcoholic steatohepatitis (ASH), liver fibrosis, liver cirrhosis, liver steatosis (fatty liver disease), liver ischemia, liver injury, Type I Diabetes, Type II Diabetes, metabolic dysfunction-associated steatotic liver disease (MASLD), Barrett's esophagus, and progressive familiar intrahepatic cholestasis (PFIC).

In some embodiments, the FXR mediated condition is metabolic dysfunction-associated steatohepatitis (MASH), primary biliary cholangitis (PBC), or primary sclerosing cholangitis (PSC). In some embodiments, the FXR mediated condition is primary biliary cholangitis (PBC) or primary sclerosing cholangitis (PSC).

In some embodiments, In some embodiments, the FXR mediated condition is MASH. In some embodiments, the FXR mediated condition is PBC. In some embodiments, the FXR mediated condition is PSC.

In some embodiments, the FXR mediated condition to be treated with the FXR agonist in combination with seladelpar is not metabolic dysfunction-associated steatohepatitis (MASH).

In some embodiments, provided is a method of treating and/or preventing a FXR mediated condition in a patient in need thereof, comprising administering to the patient a therapeutically effective amount of a FXR agonist in combination with a therapeutically effective amount of seladelpar or a pharmaceutically acceptable salt thereof, wherein the FXR mediate condition is not metabolic dysfunction-associated steatohepatitis (MASH).

In some embodiments, provided is a method of reducing at least one of cholestatic injury, inflammation, or liver fibrosis in a patient in need thereof, the method comprising administering a FXR agonist or a pharmaceutically acceptable salt thereof and a therapeutically effective amount of seladelpar or a pharmaceutically acceptable salt thereof to the patient. In some embodiments, provided is a method of reducing cholestatic injury in a patient in need thereof, the method comprising administering a FXR agonist or a pharmaceutically acceptable salt thereof and a therapeutically effective amount of seladelpar or a pharmaceutically acceptable salt thereof to the patient. In some embodiments, provided is a method of reducing inflammation in a patient in need thereof, the method comprising administering a FXR agonist or a pharmaceutically acceptable salt thereof and a therapeutically effective amount of seladelpar or a pharmaceutically acceptable salt thereof to the patient. In some embodiments, provided is a method of reducing liver fibrosis in a patient in need thereof, the method comprising administering a FXR agonist or a pharmaceutically acceptable salt thereof and a therapeutically effective amount of seladelpar or a pharmaceutically acceptable salt thereof to the patient.

### Liver Diseases

Disclosed herein is a method of treating and/or preventing a liver disease in a patient in need thereof, comprising administering to the patient a therapeutically effective amount of a FXR agonist in combination with a therapeutically effective amount of seladelpar or a pharmaceutically acceptable salt thereof.

Liver diseases are acute or chronic damages to the liver based on the duration of the disease. The liver damage may be caused by infection, injury, exposure to drugs or toxic compounds such as alcohol or impurities in foods, an abnormal build-up of normal substances in the blood, an autoimmune process, a genetic defect (such as haemochromatosis), or other unknown causes. Exemplary liver diseases include, but are not limited to, cirrhosis, liver fibrosis, metabolic dysfunction-associated steatotic liver disease (MASLD), metabolic dysfunction-associated steatohepatitis (MASH), alcoholic steatohepatitis (ASH), hepatic ischemia reperfusion injury, primary biliary cirrhosis (PBC), primary sclerosing cholangitis (PSC), and hepatitis, including both viral and alcoholic hepatitis. In some embodiments, the liver disease is selected from metabolic dysfunction-associated steatohepatitis (MASH), formally non-alcoholic steatohepatitis (NASH), primary sclerosing cholangitis (PSC), primary biliary cirrhosis (PBC), autoimmune hepatitis, alcoholic steatohepatitis (ASH), liver fibrosis, liver cirrhosis, liver steatosis (fatty liver disease), liver ischemia (liver injury in Cortellis), metabolic dysfunction-associated steatotic liver disease (MASLD), and progressive familiar intrahepatic cholestasis (PFIC).

Metabolic dysfunction-associated steatotic liver disease (MASLD), is the buildup of extra fat in liver cells that is not caused by alcohol. MASLD may cause the liver to swell (i.e., steatohepatitis), which in turn may cause scarring (i.e., cirrhosis) over time and may lead to liver cancer or liver failure. MASLD is characterized by the accumulation of fat in hepatocytes and is often associated with some aspects of metabolic syndrome (e.g., type 2 diabetes mellitus, insulin resistance, hyperlipidemia, hypertension). The frequency of this disease has become increasingly common due to consumption of carbohydrate-rich and high fat diets. A subset (~20%) of MASLD patients develop metabolic dysfunction-associated steatohepatitis (MASH).

MASH, a subtype of fatty liver disease, is the more severe form of MASLD. It is characterized by macrovesicular steatosis, balloon degeneration of hepatocytes, and/or inflammation ultimately leading to hepatic scarring (i.e., fibrosis). Patients diagnosed with MASH progress to advanced stage liver fibrosis and eventually cirrhosis. The current treatment for cirrhotic MASH patients with end-stage disease is liver transplant.

Another common liver disease is primary sclerosing cholangitis (PSC). It is a chronic or long-term liver disease that slowly damages the bile ducts inside and outside the liver. In patients with PSC, bile accumulates in the liver due to blocked bile ducts, where it gradually damages liver cells and causes cirrhosis, or scarring of the liver. Currently, there is no effective treatment to cure PSC. Many patients having PSC ultimately need a liver transplant due to liver failure, typically about 10 years after being diagnosed with the disease. PSC may also lead to bile duct cancer.

Liver fibrosis is the excessive accumulation of extracellular matrix proteins, including collagen, which occurs in most types of chronic liver diseases. Advanced liver fibrosis results in cirrhosis, liver failure, and portal hypertension and often requires liver transplantation.

In some embodiments, the liver disease is metabolic dysfunction-associated steatohepatitis (MASH), primary biliary cholangitis (PBC), or primary sclerosing cholangitis (PSC). In some embodiments, the liver disease is primary biliary cholangitis (PBC) or primary sclerosing cholangitis (PSC).

In some embodiments, In some embodiments, the liver disease is MASH. In some embodiments, the liver disease is PBC. the liver disease is PSC.

In some embodiments, the liver disease to be treated with the FXR agonist in combination with seladelpar is not metabolic dysfunction-associated steatohepatitis (MASH).

In some embodiments, provided is a method of treating and/or preventing a liver disease in a patient in need thereof, comprising administering to the patient a therapeutically effective amount of a FXR agonist in combination with a therapeutically effective amount of seladelpar or a pharmaceutically acceptable salt thereof, wherein liver disease is not metabolic dysfunction-associated steatohepatitis (MASH).

As described in Examples, it has been discovered that elevated intracellular levels of bile acids interact directly with FXR to increase the production of the pruritogenic cytokine IL-31. In other words, high levels of intracellular bile acids activate FXR-mediated production of IL-31, which is sufficient to cause pruritus. This is observed in conditions like ICP, PBC, and PSC. It is likely that IL-31 levels are elevated in other cholestatic and/or liver diseases, such as the various forms of progressive familial intrahepatic cholestasis (PFIC).

In one embodiment, the patient of the FXR mediated condition or the liver disease has been previously administered a FXR agonist and/or had pruritus. The pruritus may be FXR-induced. The FXR-induced pruritus may be due to the patient being administered an FXR agonist or due to bile acids.

In one embodiment, the FXR agonist and seladelpar or its pharmaceutically acceptable salt may be administered together in a combination formulation or in separate pharmaceutical compositions, where each inhibitor may be formulated in any suitable dosage form. In certain embodiments, the methods provided herein comprise administering separately a pharmaceutical composition comprising an FXR agonist and a pharmaceutically acceptable carrier or excipient and a pharmaceutical composition comprising seladelpar or its pharmaceutically acceptable salt and a pharmaceutically acceptable carrier or excipient. Combination formulations according to the present disclosure comprise an FXR agonist and seladelpar or its pharmaceutically acceptable salt together with one or more pharmaceutically acceptable carriers or excipients and optionally other therapeutic agents. Combination formulations containing the active ingredient may be in any form suitable for the intended method of administration.

Also disclosed herein is a method of treating pruritus, such as Farnesoid X Receptor (FXR)-induced pruritus, comprising administering to a patient in need thereof a therapeutically effective amount of seladelpar or a pharmaceutically acceptable salt thereof. The patient may be suffering from an FXR mediated condition. The FXR-induced pruritus may be due to the patient being administered an FXR agonist or due to bile acids. In some embodiments, the FXR-induced pruritus is due to the patient being administered an FXR agonist. In some embodiments, the FXR-induced pruritus is due to bile acids.

### FXR Agonists

Exemplary FXR agonists for use in the methods described herein are as follows. Specific FXR agonists as well as methods for preparing FXR agonists as described herein can be found in WO 2013/007387, WO 2017/218330, and WO 2020/150136, which publications are hereby incorporated by reference in their entirety.

In some embodiments, the FXR agonists include, but are not limited to, those described in US2014221659, US2020281911, and WO2020185685.

In some embodiments, the FXR agonist is AGN-242266, AGN-242256, ASC-42, EDP-297 (EP-024297), RDX-023, BWL-200, AKN-083, EDP-305, GNF-5120, cilofexor tromethamine (GS-9674), GS-8670, HPG-1860, IOT-022, LMB-763, obeticholic acid, Px-102, Px-103, M790, M780, M450, M-480, MET-409, MET-642, PX20606, SYHA-1805, vonafexor (EYP-001), TERN-101, TC-100, INT-2228, TQA-3526, ZG-5266, HPD-001, or alendronate.

In some embodiments, the FXR agonist is the FXR agonist is selected from the group consisting of obeticholic acid (OCA), GW4064, ASC-42, PX-104, TERN 101, cilofexor, tropifexor, GS-8670, vonafexor, EDP-305, MET-409, or a pharmaceutically acceptable salt thereof.

In some embodiments, the FXR agonist is tropifexor (LJN452), idufexor (LMB-763), AGN-242266, AGN-242256, ASC-42, EDP-297 (EP-024297), RDX-023, BWL-200, AKN-083, EDP-305, GNF-5120, cilofexor tromethamine (GS-9674), GS-8670, HPG-1860, IOT-022, LMB-763, obeticholic acid, Px-102, Px-103, M790, M780, M450, M-480, MET-409, MET-642, PX20606, SYHA-1805, vonafexor (RYP-001), TERN-101, TC-100, INT-2228, TQA-3526, ZG-5266, HPD-001 alendronate,

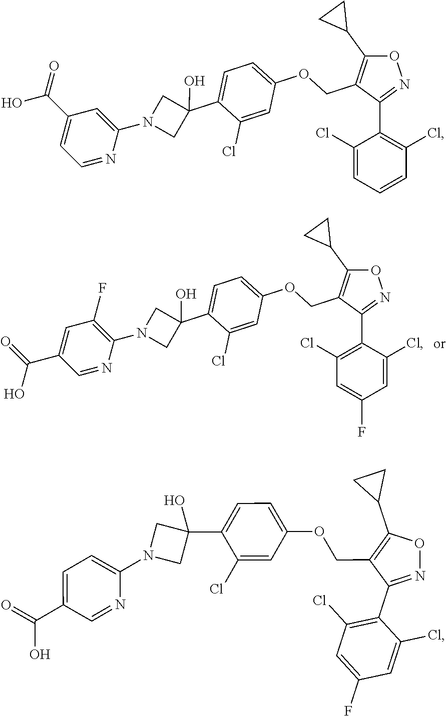 [SMILES: O=C(O)c1ccnc(N2CC(O)(c3ccc(OCc4c(-c5c(Cl)cccc5Cl)noc4C4CC4)cc3Cl)C2)c1 ; O=C(O)c1cnc(N2CC(O)(c3ccc(OCc4c(-c5c(Cl)cc(F)cc5Cl)noc4C4CC4)cc3Cl)C2)c(F)c1 ; O=C(O)c1ccc(N2CC(O)(c3ccc(OCc4c(-c5c(Cl)cc(F)cc5Cl)noc4C4CC4)cc3Cl)C2)nc1]

or a pharmaceutically acceptable salt thereof.

In some embodiments, the FXR agonist is:

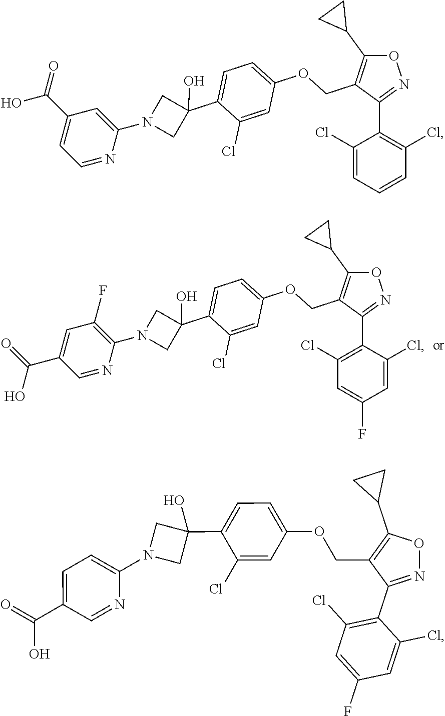 [SMILES: O=C(O)c1ccnc(N2CC(O)(c3ccc(OCc4c(-c5c(Cl)cccc5Cl)noc4C4CC4)cc3Cl)C2)c1 ; O=C(O)c1cnc(N2CC(O)(c3ccc(OCc4c(-c5c(Cl)cc(F)cc5Cl)noc4C4CC4)cc3Cl)C2)c(F)c1 ; O=C(O)c1ccc(N2CC(O)(c3ccc(OCc4c(-c5c(Cl)cc(F)cc5Cl)noc4C4CC4)cc3Cl)C2)nc1]

or a pharmaceutically acceptable salt thereof.

In some embodiments the FXR agonist is:

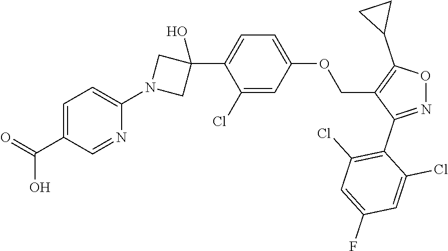 [SMILES: O=C(O)c1ccc(N2CC(O)(c3ccc(OCc4c(-c5c(Cl)cc(F)cc5Cl)noc4C4CC4)cc3Cl)C2)nc1]

or a pharmaceutically acceptable salt thereof.

In some embodiments, the FXR agonist is:

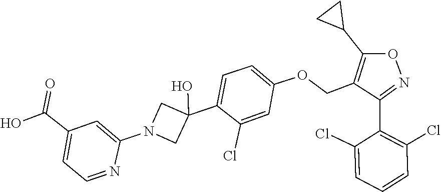 [SMILES: O=C(O)c1ccnc(N2CC(O)(c3ccc(OCc4c(-c5c(Cl)cccc5Cl)noc4C4CC4)cc3Cl)C2)c1]

or a pharmaceutically acceptable salt thereof.

In some embodiments, the FXR agonist is

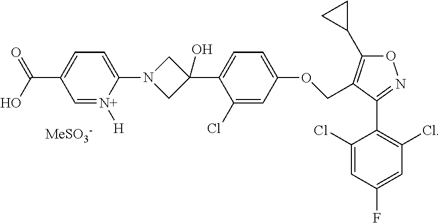 [SMILES: "MeSO3-" ; O=C(O)c1ccc(N2CC(O)(c3ccc(OCc4c(-c5c(Cl)cc(F)cc5Cl)noc4C4CC4)cc3Cl)C2)[nH+]c1]

In some embodiments, the FXR agonist is cilofexor or a pharmaceutically acceptable salt thereof.

In some embodiments, the FXR agonist is GS-8670 or a pharmaceutically acceptable salt thereof.

In some embodiments, the FXR agonist is obeticholic acid (OCA) or a pharmaceutically acceptable salt thereof.

In some embodiments, the FXR agonist is GW4064 or a pharmaceutically acceptable salt thereof.

In some embodiments, the FXR agonist is ASC-42 or a pharmaceutically acceptable salt thereof.

In some embodiments, the FXR agonist is PX-104 or a pharmaceutically acceptable salt thereof.

In some embodiments, the FXR agonist is TERN 101 or a pharmaceutically acceptable salt thereof.

In some embodiments, the FXR agonist is tropifexor or a pharmaceutically acceptable salt thereof.

In some embodiments, the FXR agonist is vonafexor or a pharmaceutically acceptable salt thereof.

In some embodiments, the FXR agonist is idufexor or a pharmaceutically acceptable salt thereof.

In some embodiments, the FXR agonist is EDP-305 or a pharmaceutically acceptable salt thereof.

In some embodiments, the FXR agonist is MET-409 or a pharmaceutically acceptable salt thereof.

Also included are optical isomers, racemates, or other mixtures thereof of the compounds described herein or pharmaceutically acceptable salts or a mixture thereof. In those situations, the single enantiomer or diastereomer, i.e., optically active form, can be obtained by asymmetric synthesis or by resolution. Resolution can be accomplished, for example, by conventional methods such as crystallization in the presence of a resolving agent, or chromatography, using for example, a chiral high pressure liquid chromatography (HPLC) column.

Compositions provided herein can include a compound described herein or pharmaceutically acceptable salts, isomer, or a mixture thereof may include racemic mixtures, or mixtures containing an enantiomeric excess of one enantiomer or single diastereomers or diastereomeric mixtures. All such isomeric forms of these compounds are expressly included herein the same as if each and every isomeric form were specifically and individually listed.

A composition comprising a mixture of enantiomers (or diastereomers) of a compound described herein or a pharmaceutically acceptable salt thereof, is also provided herein. In some embodiments, the composition comprises a single enantiomer of the compound and is substantially free of the other enantiomer. In certain embodiments, the compound of Formula 1A or 1B (or another Formula as described herein) contains one or more additional stereogenic atom(s) (e.g., at R1 and/or R3). In such instances, the composition may contain a mixture of diastereomers. In some embodiments, the composition comprises a single enantiomer of the compound and is substantially free (i.e., having less than or about 40%, 30%, 25%, 20%, 15%, 10%, 5%, 1%, 0.05%, or 0.01%) of one or more diastereomers.

### Seladelpar

Seladelpar (International Nonproprietary Name-INN), having the following structure, has the chemical name [4-({(2R)-2-ethoxy-3-[4-(trifluoromethyl)phenoxy]propyl}sulfanyl)-2-methylphenoxy]acetic acid.

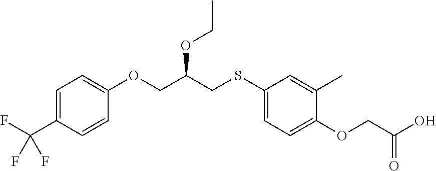 [SMILES: CCO[C@H](COc1ccc(C(F)(F)F)cc1)CSc1ccc(OCC(=O)O)c(C)c1]

Seladelpar and its synthesis, formulation, and use are disclosed in, for example, U.S. Pat. Nos. 7,301,050, 7,635,718, and 8,106,095 (compound 15 in Table 1, Example M, claim 14). Lysine (L-lysine) salts of seladelpar and related compounds are disclosed in U.S. Pat. No. 7,709,682.

Seladelpar is an orally active, potent agonist of peroxisome proliferator-activated receptor-6 (PPARδ). It is specific (>600-fold and >2500-fold compared with peroxisome proliferator-activated receptor-α and peroxisome proliferator-activated receptor-γ receptors) to PPARδ. PPARδ activation stimulates fatty acid oxidation and utilization, improves plasma lipid and lipoprotein metabolism, glucose utilization, and mitochondrial respiration, and preserves stem cell homeostasis. According to U.S. Pat. No. 7,301,050, PPARδ agonists, such as seladelpar, are suggested to treat PPARδ-mediated conditions, including “diabetes, cardiovascular diseases, Metabolic X syndrome, hypercholesterolemia, hypo-high density lipoprotein (HDL)-cholesterolemia, hyper-low density protein (LDL)-cholesterolemia, dyslipidemia, atherosclerosis, and obesity,” with dyslipidemia said to include hypertriglyceridemia and mixed hyperlipidemia. Seladelpar may be provided as seladelpar lysine (L-lysine) salt dihydrate.

Pharmacological activity leading to therapeutic effects includes inhibition of bile acid synthesis through activation of PPARδ, which is a nuclear receptor expressed in most tissues, including the liver. Published studies show that PPARδ activation by seladelpar reduces bile acid synthesis through Fibroblast Growth Factor 21 (FGF21)-dependent downregulation of CYP7A1, the key enzyme for the synthesis of bile acids from cholesterol,

Seladelpar, as a L-lysine salt dihydrate, has been approved for at a dose equivalent to 10 mg/day for the treatment of primary biliary cholangitis (PBC). Seladelpar studied at oral doses equivalent to 50 and 100 mg/day of seladelpar in mixed dyslipidemia; at doses equivalent to 10, 20, and 50 mg/day of seladelpar in metabolic dysfunction-associated steatohepatitis; and at doses equivalent to 2, 5, 10, 50, and 200 mg/day of seladelpar in PBC.

Seladelpar is also associated with decreases in the pruritogenic cytokine Interleukin-31 (IL-31). U.S. Application Publication No. 2019/0105291 and PCT International Publication No. WO 2019/067373 disclose the treatment of cholestatic pruritus with seladelpar and its salts.

Both seladelpar and FXR agonists are reported to reduce bile acid levels by downregulating CYP7A1. However, as described in Examples, it has been shown that FXR agonists paradoxically increase pruritus due to their transcriptional activation of FXR-mediated expression of IL-31, resulting in elevated IL-31 levels and pruritus. In contrast, it has been discovered that, in primary human hepatocyte cultures, seladelpar reduced IL-31 production even in the presence of excess FXR agonism, suggesting a novel additional mechanism of action for seladelpar that is independent of bile acid production. Thus, while seladelpar can decrease bile acid production and thereby lower FXR activity, it can also negatively regulate IL-31 expression, even in the continuous presence of FXR activation.

Unless the context requires otherwise, reference to seladelpar, and reference to “a compound that is seladelpar or a salt thereof,” is a reference both to seladelpar itself and to its salts. An amount of a seladelpar salt that is “equivalent to” a particular amount of seladelpar refers to that amount of the salt that is the particular amount multiplied by the ratio of the formula weight of the salt to the formula weight of seladelpar. For example, if seladelpar L-lysine salt dihydrate is being used, since the formula weight of seladelpar L-lysine salt dihydrate is about 1.41 times the formula weight of seladelpar, an amount of about 14.1 mg/day of seladelpar L-lysine salt dihydrate will be equivalent to an amount of 10 mg/day of seladelpar.

### Additional Therapeutic Agents

In some embodiments, a compound of the disclosure, or a pharmaceutically acceptable salt thereof, is co-administered with one or more agents useful for the treatment and/or prophylaxis of a heptatologic condition, such as MASLD, or MASH. Non-limiting examples of such agents include glucagon-like peptide-1 receptor agonists (GLP-1 RA, e.g., liraglutide, semaglutide, tirzepatide), sodium-glucose cotransporter-2 inhibitor (SGLT2i), thiazolidinediones (pioglitazone) and vitamin E.

In some embodiments, a compound of the disclosure, or a pharmaceutically acceptable salt thereof, is co-administered with one or more agents useful for the treatment and/or prophylaxis of a heptatologic condition, such as PBC or PSC. Non-limiting examples of such agents include ursodeoxycholic acid or obeticholic acid.

In some embodiments, a compound or compounds (i.e., a FXR agonist and/or seladelpar or pharmaceutically acceptable salt thereof) of the disclosure is co-administered with one or more (e.g., one, two, three, or four) additional therapeutic agents. In some embodiments, the additional therapeutic agent includes an agent useful for modulating, treating, or preventing inflammation, such as a 1,3 beta glucanosyltransferase GAS2 modulator, 11-Beta hydroxysteroid dehydrogenase 1 inhibitor, 11-Beta hydroxysteroid dehydrogenase inhibitor, 12-Lipoxygenase inhibitor, 17 beta hydroxysteroid dehydrogenase 13 inhibitor, 2-Acylglycerol O-acyltransferase 2 inhibitor, 3 Ketoacyl CoA thiolase inhibitor, 40S ribosomal protein S4 stimulator, 5-HT 2a receptor agonist, 5-HT 2a receptor antagonist, 5-Lipoxygenase inhibitor, 6-Phosphofructokinase inhibitor, ACAT inhibitor, ACAT-2 inhibitor, Acetyl CoA carboxylase inhibitor, Acetyl CoA carboxylase-1 inhibitor, Acetyl CoA carboxylase-2 inhibitor, Acidic mammalian chitinase inhibitor, ACMSD gene inhibitor, Actin antagonist, Activin type-IIB receptor antagonist, Adenosine A1 receptor antagonist, Adenosine A2a receptor antagonist, Adenosine A3 receptor agonist, Adenosine A3 receptor antagonist, Adenosine kinase inhibitor, Adenylyl cyclase associated protein 1 inhibitor, Adiponectin receptor agonist, Adiponectin receptor modulator, Adiponectin receptor-1 agonist, Adiponectin receptor-2 agonist, Adiponutrin inhibitor, Adipose triglyceride lipase stimulator, Adrenergic receptor antagonist, Advanced glycosylation product receptor antagonist, AKT protein kinase stimulator, Albumin agonist, Albumin modulator, Aldosterone antagonist, Alkaline phosphatase modulator, Alpha 1 antitrypsin stimulator, Alpha 1 proteinase inhibitor, Alpha-amylase inhibitor, Alpha-glucosidase inhibitor, Alstrom syndrome protein 1 modulator, Aminopeptidase N modulator, Aminotransferase stimulator, AMP activated protein kinase alpha 2 stimulator, AMP activated protein kinase modulator, AMP activated protein kinase stimulator, Amylin receptor agonist, Amylin receptor antagonist, Amylin receptor modulator, Amyloid protein deposition inhibitor, Anaphase promoting complex subunit modulator, Androgen receptor agonist, Angiotensin converting enzyme 2 inhibitor, Angiotensin converting enzyme gene modulator, Angiotensin I receptor antagonist, Angiotensin II AT-1 receptor antagonist, Angiotensin II receptor antagonist, ANP agonist, AP1 transcription factor modulator, APOA1 gene stimulator, APOC3 gene inhibitor, APOE gene stimulator, Arginine deiminase stimulator, Arrestin domain containing protein 1 modulator, Aryl hydrocarbon receptor agonist, Asialoglycoprotein receptor 1 modulator, ATP citrate lyase inhibitor, Autophagy protein inhibitor, Autophagy protein modulator, Autophagy protein stimulator, Axin modulator, Bax protein stimulator, BC ketoacid dehydrogenase kinase inhibitor, Beta 2 adrenoceptor agonist, Beta 3 adrenoceptor agonist, Beta amyloid modulator, Beta secretase 2 inhibitor, Beta-catenin inhibitor, Bifunctional aminoacyl tRNA synthetase inhibitor, B-lymphocyte antigen CD19 modulator, B-lymphocyte stimulator ligand inhibitor, Bone morphogenetic protein-11 ligand, Bone morphogenetic protein-2 ligand inhibitor, Bone morphogenetic protein-4 ligand inhibitor, Bone morphogenetic protein-7 ligand, Bromodomain containing protein 2 inhibitor, Bromodomain containing protein 4 inhibitor, Cak tyrosine kinase receptor inhibitor, Calcineurin inhibitor, Calcitonin receptor agonist, Calcium channel inhibitor L-type, Calcium channel stimulator, Calpain inhibitor, Calpain-I inhibitor, Calpain-II inhibitor, Calpain-IX inhibitor, CAM kinase II inhibitor, Cannabinoid CB1 receptor antagonist, Cannabinoid CB1 receptor inverse agonist, Cannabinoid CB1 receptor modulator, Cannabinoid CB2 receptor modulator, Casein kinase II alpha inhibitor, Caspase inhibitor, Caveolin 1 inhibitor, CCAAT enhancer binding protein beta modulator, CCK receptor antagonist, CCL26 gene inhibitor, CCR2 chemokine antagonist, CCR3 chemokine modulator, CCR5 chemokine antagonist, CD122 modulator, CD158a antagonist, CD158b1 antagonist, CD158b2 antagonist, CD2 antagonist, CD3 antagonist, CD3 modulator, CD4 agonist, CD4 modulator, CD40 ligand inhibitor, CD40 ligand receptor antagonist, CD47 antagonist, CD80 antagonist, CDGSH iron sulfur domain protein modulator, Cell adhesion molecule inhibitor, Chitinase inhibitor, Chitotriosidase 1 inhibitor, Cholecystokinin CCK2 receptor antagonist, Chymase inhibitor, CIDEB gene inhibitor, Claudin 1 inhibitor, C-myc binding protein inhibitor, Collagen gene inhibitor, Collagen I antagonist, Collagen I modulator, Collagen III agonist, Collagen modulator, Complement C1Q TNF-related protein modulator, Connective tissue growth factor ligand inhibitor, COT protein kinase inhibitor, CREB binding protein inhibitor, CRF-2 receptor antagonist, Cryptochrome modulator, CTGF gene inhibitor, CXCL10 gene inhibitor, CXCL11 gene inhibitor, CXCL9 gene inhibitor, CXCR1 chemokine antagonist, CXCR2 chemokine antagonist, CXCR3 chemokine antagonist, CXCR4 chemokine antagonist, CXCR7 chemokine agonist, Cyclin-dependent kinase-4 inhibitor, Cyclin-dependent kinase-6 inhibitor, Cyclooxygenase 2 inhibitor, Cyclooxygenase inhibitor, Cytochrome P450 2E1 inhibitor, Cytochrome P450 3A4 inhibitor, Cytochrome P450 7A1 inhibitor, Cytochrome P450 reductase inhibitor, Cytotoxic T-lymphocyte protein-4 stimulator, DGAT2 gene inhibitor, Diacylglycerol O acyltransferase 1 inhibitor, Diacylglycerol O acyltransferase 2 inhibitor, Dihydroceramide delta 4 desaturase inhibitor, Dihydroorotate dehydrogenase inhibitor, Dipeptidyl peptidase IV inhibitor, DNA cytosine-5 methyltransferase 1 inhibitor, DNA gyrase inhibitor, DNA methyltransferase inhibitor, Dopamine D2 receptor agonist, DYRK-1 alpha protein kinase inhibitor, E3 ubiquitin protein ligase COP1 modulator, E3 ubiquitin protein ligase ZNRF2 modulator, Ectonucleotide pyrophosphatase-PDE-2 inhibitor, EIF-2 alpha protein kinase stimulator, Endoglin inhibitor, Endothelial nitric oxide synthase stimulator, Endothelin ET-A receptor antagonist, Endothelin ET-B receptor antagonist, Eotaxin 2 ligand inhibitor, Eotaxin ligand inhibitor, Epha4 tyrosine kinase receptor stimulator, Ephb2 tyrosine kinase receptor inhibitor, Ephrin B2 ligand inhibitor, Epidermal growth factor receptor agonist, Epidermal growth factor receptor antagonist, Epidermal growth factor receptor modulator, Epoxide hydrolase inhibitor, Erbb2 tyrosine kinase receptor inhibitor, Erbb2 tyrosine kinase receptor modulator, Erythropoietin receptor agonist, Estradiol 17 beta dehydrogenase 1 inhibitor, Estrogen related receptor alpha modulator, Exendin 4 ligand, Extracellular signal related kinase modulator, Factor IIa antagonist, Farnesoid X receptor agonist, Farnesoid X receptor modulator, Fatty acid synthase inhibitor, FGF binding protein 3 stimulator, FGF receptor agonist, FGF-1 ligand, FGF1 receptor agonist, FGF19 gene stimulator, FGF-19 ligand, FGF21 gene modulator, FGF-21 ligand, FGF-21 ligand inhibitor, FGF-21 ligand modulator, FGF-3 ligand, FGFR1 oncogene modulator, FOXO1 transcription factor inhibitor, Free fatty acid receptor 1 agonist, Free fatty acid receptor 2 agonist, Free fatty acid receptor 3 agonist, Fructose 1,6 biphosphatase inhibitor, Fyn tyrosine kinase inhibitor, G protein coupled receptor 142 agonist, GABA A receptor antagonist, GABA A receptor modulator, GABA receptor agonist, GABA receptor modulator, Galanin receptor antagonist, Galectin-3 inhibitor, Gamma-secretase inhibitor, Gastric inhibitory peptide ligand, Gastric inhibitory polypeptide receptor agonist, Gastric inhibitory polypeptide receptor modulator, GCGR gene inhibitor, GDF-15 ligand, GDF-8 antagonist, GDNF family receptor alpha like agonist, GDNF receptor modulator, GHRH receptor agonist, Glucagon ligand, Glucagon receptor agonist, Glucagon receptor antagonist, Glucagon-like peptide 1 gene modulator, Glucagon-like peptide 1 receptor agonist, Glucagon-like peptide 1 receptor antagonist, Glucagon-like peptide 1 receptor modulator, Glucagon-like peptide 2 receptor agonist, Glucocorticoid receptor agonist, Glucocorticoid receptor antagonist, Glucokinase inhibitor, Glucokinase stimulator, Glucose 6-phosphate 1-dehydrogenase inhibitor, Glutamate decarboxylase modulator, Glutaminyl peptide cyclotransferase inhibitor, Glycogen synthase kinase-3 beta inhibitor, Glycosidase inhibitor, GP IIb IIIa antagonist, G-protein coupled bile acid receptor 1 agonist, G-protein coupled receptor 120 agonist, G-protein coupled receptor 84 antagonist, G-protein coupled receptor-119 agonist, G-protein coupled receptor-39 antagonist, Guanylate cyclase stimulator, Hedgehog protein inhibitor, Heparanase inhibitor, Hepatitis B structural protein inhibitor, Hepatitis C virus protein NS5A inhibitor, Hepatocyte growth factor receptor agonist, Hepatocyte growth factor receptor modulator, Hepatocyte nuclear factor-4 receptor modulator, Histone deacetylase inhibitor, Histone deacetylase-1 inhibitor, Histone deacetylase-2 inhibitor, Histone deacetylase-3 inhibitor, Histone lysine methyltransferase EHMT2 inhibitor, HIV gp41 protein modulator, HIV-1 gp120 protein inhibitor, HLA antigen inhibitor, HLA class I antigen A-2 alpha modulator, HLA class II antigen inhibitor, HLA class II antigen modulator, HMG CoA reductase inhibitor, Hormone sensitive lipase stimulator, HSD17B13 gene inhibitor, HSD17B13 gene modulator, Hydrolase inhibitor, IGF binding protein-3 inhibitor, I-kappa B kinase epsilon inhibitor, IL-1 receptor antagonist, IL-10 receptor agonist, IL-11 receptor antagonist, IL-12 receptor antagonist, IL-17 antagonist, IL-2 receptor agonist, IL-22 agonist, IL-8 receptor antagonist, Ileal sodium bile acid cotransporter inhibitor, Ileal sodium bile acid cotransporter modulator, Immunoglobulin G1 agonist, Immunoglobulin gamma Fc receptor agonist, Inducible nitric oxide synthase inhibitor, INHBE gene inhibitor, INS gene modulator, Insulin degrading enzyme inhibitor, Insulin induced gene protein stimulator, Insulin ligand, Insulin receptor agonist, Insulin receptor substrate-2 stimulator, Insulin sensitizer, Insulin-like growth factor 1 receptor agonist, Insulin-like growth factor 1 receptor antagonist, Insulin-like growth factor 1 receptor modulator, Integrin alpha-V/beta-1 antagonist, Integrin alpha-V/beta-3 antagonist, Integrin alpha-V/beta-3 modulator, Integrin alpha-V/beta-5 modulator, Integrin alpha-V/beta-6 antagonist, Integrin antagonist, Interferon alpha 2 ligand, Interferon alpha ligand, Interferon alpha ligand inhibitor, Interferon gamma ligand, Interleukin 11 ligand modulator, Interleukin 17 ligand inhibitor, Interleukin 18 ligand inhibitor, Interleukin-1 alpha ligand inhibitor, Interleukin-1 beta ligand inhibitor, Interleukin-2 ligand, IRE1 protein kinase inhibitor, Islet amyloid polypeptide inhibitor, Jak1 tyrosine kinase inhibitor, Jak2 tyrosine kinase inhibitor, Jak3 tyrosine kinase inhibitor, Jun N terminal kinase inhibitor, Jun N terminal kinase-1 inhibitor, KCNA voltage-gated potassium channel-3 modulator, Kelch like ECH associated protein 1 inhibitor, Kelch like ECH associated protein 1 modulator, Ketohexokinase inhibitor, Killer cell Ig like receptor antagonist, Klotho beta stimulator, LanC like protein 2 modulator, Leptin receptor agonist, Leukocyte elastase inhibitor, Leukotriene A4 hydrolase inhibitor, Leukotriene CysLT1 receptor antagonist, Leukotriene D4 antagonist, Lipase inhibitor, Liver X receptor alpha inverse agonist, Liver X receptor antagonist, Liver X receptor beta inverse agonist, Liver X receptor modulator, LKB1 protein kinase stimulator, LOXL2 gene inhibitor, Lymphocyte function antigen-3 receptor modulator, Lyn tyrosine kinase stimulator, Lysophosphatidate-1 receptor antagonist, Lysyl oxidase homolog 2 inhibitor, Lysyl tRNA synthetase inhibitor, Macrophage mannose receptor 1 modulator, MAdCAM modulator, MAFA gene stimulator, MALT protein 1 inhibitor, MAP kinase 4 inhibitor, MARC1 gene inhibitor, Mas-related G-protein receptor D agonist, Matrix metalloprotease inhibitor, MEKK-5 protein kinase inhibitor, Membrane copper amine oxidase inhibitor, Menin inhibitor, Metalloprotease-9 stimulator, Methionine aminopeptidase-2 inhibitor, Mitochondrial carrier family modulator, Mitochondrial pyruvate carrier 2 inhibitor, Mitochondrial pyruvate carrier inhibitor, Mono ADP ribosyltransferase sirtuin-6 inhibitor, Mono ADP ribosyltransferase sirtuin-6 modulator, Motile sperm domain protein 2 inhibitor, MST-1 protein kinase inhibitor, mTOR complex 1 inhibitor, mTOR inhibitor, Myeloid lymphoid leukemia protein inhibitor, Myeloperoxidase inhibitor, NACHT LRR PYD domain protein 3 inhibitor, NAD-dependent deacetylase sirtuin stimulator, NAD-dependent deacetylase sirtuin-1 inhibitor, NAD-dependent deacetylase sirtuin-1 stimulator, NADH quinone oxidoreductase stimulator, NADPH oxidase 1 inhibitor, NADPH oxidase 4 inhibitor, NADPH oxidase inhibitor, Natriuretic peptide receptor B agonist, Nek-7 protein kinase inhibitor, Neuropeptide Y2 receptor agonist, NFE2L2 gene inhibitor, Nicotinamide N methyltransferase inhibitor, Nicotinamide phosphoribosyltransferase inhibitor, NK1 receptor antagonist, NLRP3 gene inhibitor, NMDA receptor antagonist, Notch antagonist, NOTCH1 gene inhibitor, NR1H3 gene inhibitor, Nuclear erythroid 2-related factor 2 stimulator, Nuclear factor kappa B modulator, Nuclear receptor 5A2 agonist, Nuclear receptor 5A2 modulator, Nucleoside reverse transcriptase inhibitor, Opioid receptor kappa agonist, Opioid receptor mu antagonist, Ornithine decarboxylase inhibitor, Orphan nuclear hormone receptor NR4A1 antagonist, Orphan nuclear receptor NURR1 agonist, OX-40 receptor antagonist, Oxyntomodulin ligand, P2X7 purinoceptor modulator, P2Y12 purinoceptor antagonist, P2Y13 purinoceptor stimulator, p38 MAP kinase inhibitor, PACAP ligand, PACAP type I receptor agonist, Palmitoyl protein thioesterase 1 inhibitor, Pancreatic beta cell growth factor modulator, Patatin like phospholipase protein modulator, PAX4 gene stimulator, PDE 3 inhibitor, PDE 4 inhibitor, PDGF receptor beta modulator, PDX1 gene stimulator, Peptide YY ligand, Peptide YY ligand modulator, Peptidyl-prolyl cis-trans isomerase A inhibitor, Peptidyl-prolyl cis-trans isomerase B inhibitor, Peptidyl-prolyl cis-trans isomerase D inhibitor, Phenylalanine hydroxylase stimulator, Phosphatase stimulator, Phosphatidylinositol 3 kinase subunit 3 inhibitor, Phospholipase C inhibitor, Phosphoric diester hydrolase inhibitor, Picolinate carboxylase inhibitor, Plasma retinol binding protein inhibitor, PNPLA3 gene inhibitor, PNPLA3 gene modulator, Potassium channel inhibitor, PPAR agonist, PPAR alpha agonist, PPAR delta agonist, PPAR delta antagonist, PPAR gamma agonist, PPAR gamma coactivator 1-alpha stimulator, PPAR gamma modulator, PPAR gamma partial agonist, PPAR modulator, Pregnane X receptor agonist, Programmed cell death ligand 1, Programmed cell death ligand 1 modulator, Programmed cell death protein 1 inhibitor, Proprotein convertase PC9 inhibitor, Proprotein convertase PC9 modulator, Protease-activated receptor-2 antagonist, Proteasome beta-8 subunit modulator, Proteasome inhibitor, Protein cereblon modulator, Protein disulfide isomerase A4 inhibitor, Protein kinase C alpha modulator, Protein NOV homolog modulator, Protein tyrosine phosphatase-1B inhibitor, Protein tyrosine phosphatase-2C inhibitor, PTGS2 gene inhibitor, PTPN1 gene inhibitor, Pyruvate dehydrogenase kinase inhibitor, Regenerating islet-derived protein 3a stimulator, Relaxin agonist, Relaxin receptor 1 agonist, Remodeling and spacing factor 1 inhibitor, Renin inhibitor, Resistin ligand inhibitor, Retinoic acid receptor beta agonist, Retinoic acid receptor gamma antagonist, Retinoid X receptor alpha agonist, Retinoid X receptor modulator, Rev protein modulator, Rho associated protein kinase 2 inhibitor, Ribosomal protein S6 kinase-1 inhibitor, RNA polymerase inhibitor, Secretin antagonist, Seprase inhibitor, Seprase modulator, Serine protease inhibitor, Serine threonine protein kinase 25 inhibitor, Serine threonine protein kinase TBK1 inhibitor, Serpin B13 inhibitor, SERPINA1 gene inhibitor, Serum protein modulator, Serum response factor inhibitor, SIRT6 gene stimulator, SMAD inhibitor, SOD2 gene stimulator, Sodium bile acid cotransporter inhibitor, Sodium glucose transporter-1 inhibitor, Sodium glucose transporter-2 inhibitor, Solute carrier family inhibitor, Somatostatin 2 receptor antagonist, Somatostatin 5 receptor antagonist, Sphingolipid delta 4 desaturase DES1 inhibitor, Sphingosine kinase 1 inhibitor, Sphingosine-1-phosphate receptor-1 antagonist, Sphingosine-1-phosphate receptor-4 antagonist, SREBP cleavage activating protein inhibitor, SREBP transcription factor 1 inhibitor, SREBP transcription factor 2 inhibitor, SREBP1C gene inhibitor, STAT-1 modulator, STAT-3 inhibitor, Stearoyl CoA desaturase-1 inhibitor, Stellate protein kinase inhibitor, Subtilisin inhibitor, Sucrose alpha-glucosidase inhibitor, Synaptic vesicular amine transporter inhibitor, Syncytin 1 inhibitor, T cell receptor agonist, T cell receptor modulator, T cell surface glycoprotein CD28 inhibitor, TACE inhibitor, Tafazzin stimulator, Taste receptor type 2 agonist, Tau protein inhibitor, T-cell surface glycoprotein CD1d modulator, T-cell surface glycoprotein CD8 modulator, T-cell transcription factor NFAT modulator, Tec tyrosine kinase inhibitor, Telomerase stimulator, TERT gene modulator, TGF beta 1 ligand inhibitor, TGF beta 1 ligand modulator, TGF beta 3 ligand inhibitor, TGF beta ligand inhibitor, TGF beta ligand modulator, TGF beta receptor antagonist, TGFB1 gene inhibitor, Thioredoxin interacting protein inhibitor, Thioredoxin reductase inhibitor, Thrombospondin-1 ligand inhibitor, Thromboxane A2 antagonist, Thyroid hormone receptor agonist, Thyroid hormone receptor beta agonist, Tissue transglutaminase inhibitor, TLR antagonist, TLR modulator, TLR-2 antagonist, TLR-4 agonist, TLR-4 antagonist, TLR-9 antagonist, TNF alpha induced protein 3 stimulator, TNF alpha ligand inhibitor, TNF antagonist, TNF binding agent, TNF related apoptosis inducing ligand, Topoisomerase IV inhibitor, TRAIL receptor agonist, TRAIL-1 receptor modulator, TRAIL-2 receptor antagonist, Transcriptional regulator protein BACH1 modulator, TRIM33 gene modulator, Tripartite motif containing protein 72 inhibitor, TRP cation channel V1 antagonist, Trypsin inhibitor, Tumor necrosis factor 13C receptor antagonist, Tumor necrosis factor 14 ligand inhibitor, Type I TNF receptor antagonist, Tyrosine kinase receptor modulator, Tyrosine phosphatase substrate 1 inhibitor, Ubiquitin ligase modulator, Uncoupling protein modulator, Urate anion exchanger 1 inhibitor, Urocortin II ligand, Vasopressin V1 receptor agonist, Vasopressin VIA partial agonist, Vasopressin V1a receptor agonist, Vasopressin V1a receptor antagonist, Vasopressin V2 receptor antagonist, VEGF receptor modulator, VEGF-A ligand inhibitor, VEGF-B ligand inhibitor, Vimentin inhibitor, Vitamin D3 receptor agonist, Wnt ligand, Wnt ligand modulator, Wnt-1 induced signal pathway protein 1 inhibitor, Xanthine oxidase inhibitor, ZSCAN4 gene stimulator, or 1 Aminocyclopropane carboxyl synthase inhibitor.

In some embodiments, an additional therapeutic agent includes one or more of [18F]-TZ-Z-09591, [68Ga]Ga-DO3A-SOI-GCG, 1,5-bisphenylpyrazole derivative, 11betaHSD1 inhibitors, 4P Exenatide, 5-aminolevulinic acid hydrochloride+sodium ferrous citrate, 68Ga-BOT1712, A-2906, A-4368, AAA-10, AAV8-FGF19 variant M70, AAV-Ex-4, AB-004, AB-03, AB-04, ABA-201, abatacept, ABP2111-Na, ABP-6016, AC-261066, acarbose, acarbose+metformin, ACE-1332, acetylcysteine, ACQT-1001, ACQT-1127, ACT-777991, AD-214, AD-219, AD-222, AD-301, adalimumab, ADC-001, ADD-16, ADi-100, adiponectin agonists, adipose-derived stem cell therapy, ADO-09, AdoShell Islets, ADPO-002, ADPT-02, ADR-001, AER-501, AER-601, AG-019, AGEX-BAT1, AGN-242266, AGS-500, AGTX-2003, AGTX-2004, AKS-107, AKS-440, aldafermin, aldesleukin, ALD-R491, ALE-C04, ALE-F02, ALF-301, ALF-5755, ALG-055009, allogeneic blood-derived CD34-positive adult pluripotent stem cells, allogeneic human islets of Langerhans, allogeneic human pancreatic islets of Langerhans, allogeneic human umbilical cord mesenchymal stem cells, allogeneic mesenchymal stem cell therapy, allogeneic UC-MSC therapy, allogeneic umbilical cord mesenchymal stem cells, allogenic umbilical cord-derived mesenchymal stem cell therapy, ALN-KHK, ALN-PNP, alogliptin, alogliptin benzoate+metformin hydrochloride, alogliptin benzoate+metformin hydrochloride XR, alogliptin benzoate+pioglitazone hydrochloride, alpha glucosidase inhibitors, alpha-1 antitrypsin, alpha-v/beta-6 integrin inhibitors, ALS-L1023, ALT-100, ALTSuLIN, ALY688-SR, AM-510, AMA-002, AMP-activated protein kinase inhibitors, AMPK activators, AMS-III-1086, AMTX-100, AMTX-100, amyloid protein deposition inhibitor, anagliptin, anagliptin+metformin, AND-9, ANG-4201, antagomiR-144, anti-BMP-2/4 monoclonal antibodies, anti-CXCR3 mAb, anti-F1 gene therapy, anti-fibrotic macrophage cell therapy, anti-TAGE monoclonal antibody, anti-VEGF-B antibody, Antral, AP-025, AP-026, apabetalone, APB-R3, APH-012, aprocitentan, APX-311, AR-882, ARO-HSD, ARO-INHBE, ARO-PNPLA3, AS-100283, AS-1501, ASC-41, ASC-47, aspirin trelamine hydrochloride, AT-247, AT-278, AT-299, ATB-1013, Atenas, ATGC-300A, atorvastatin+metformin, ATR-127, ATR-258, Atrosimab, ATX-304, autologous bone marrow mononuclear cells, autologous leukocyte cell therapy, autologous tolerogenic dendritic cell therapy, autologous tolerogenic dendritic cell vaccine, autologous T-reg cell therapy, AUX-101, AVD-1001, avenciguat, AVM-0703, AVO-101, AVO-1681, AVT-001, AX-0601, AX-0810, AX-2911, AXA-1125, AZD-1656, AZD-1705, AZD-2389, AZD-2693, AZD-9550, azemiglitazone potassium, B-1344, B-1654, baricitinib, basal insulin, BBT-877, BC-0306, BCKDK inhibitor, BEBT-503, BEBT-508, beinaglutide, belapectin, belimumab, bempedoic acid, berberine chloride, berberine ursodeoxycholate, bermekimab, bersacapavir, bersiporocin dihydrochloride, bertilimumab, bexagliflozin, bexotegrast, Bezafibrate, bezafibrate+obeticholic acid, BF-114, BG-148, BGM-0504, BGT-002, BHD-1028, BI-3006337, BI-3231, BI-765423, BIIB-110, BioChaperone AsPram, BioChaperone Combo, BL-001, BLD-2660, BLD-3051, BLR-200, BMN-255, BMS-687681, BMS-820132, BNC-1602, BOS-580, BOT-1712-IFNg-SD, BP-103, BR-1019, BR-3003, BR-3005, BRL-186616, BRL-191118, BRL-201009, bromocriptine, BRS-201, BS-1801, BTI-320, budesonide, BX-002, BX-003, BXT-102, BX-U004, C-455, cadisegliatin, cagrilintide, cagrilintide+semaglutide, canagliflozin, canagliflozin+metformin IR, canagliflozin+metformin XR, canakinumab, cannabidiol, cannabinoid 1 receptor antagonists, CAR Treg-cell therapy, CB-0406, CB-4211, CB5138-3, Cbi-Ex4, CBL-514, CBW-511, CC-90001, CCX-872, CD4 antigen modulator/CD8 antigen modulator/T cell receptor modulator, celastrol, cell therapy, Cellgram-LC, CELZ-001, CELZ-100, CELZ-101, CELZ-201, cenicriviroc, CGT-8012, chiglitazar, CHIN-117, CHM-273S, cibinetide, CIDEB siRNA therapy, cilofexor+firsocostat, cilofexor tromethamine, GS-8670, citric acid monohydrate, CJC-1134-PC, CKD-371, CKD-378, CKD-379, CKD-383, CKD-386, CKD-389, CKD-393, CKD-398, class I HDAC inhibitors, CLBS-03, clesacostat+ervogastat, CM-101, CM-272, CNP-103, CNP-104, cofrogliptin, colesevelam, collagen I translation inhibitors (liver fibrosis), Anima Biotech, corticotropin releasing factor receptor-2 inhibitors, CPL-207-280, CRB-486, CS-0159, CS-17919, CS-27109, CT-02, CT-388, CT-868, CT-996, CTX-211, CUD-005, cudetaxestat sodium, CUE-301, CVB1 vaccine, CVI-301, CXCL9/CXCL10/CXCL11 gene therapy, CYRS-101, CYRS-MD02, CYTX-100, D4-201-01, D-745, D-759, DA-1241, DA-1726, DA-302165S, DA-5221-T, dalpiciclib, danuglipron, dapagliflozin+glimepiride+metformin hydrochloride, dapagliflozin+linagliptin, dapagliflozin+metformin, dapagliflozin+sitagliptin, dapagliflozin+sitagliptin+metformin hydrochloride, dapagliflozin+teneligliptin, dapagliflozin+vildagliptin, dapagliflozin citrate, dapagliflozin ER, dapagliflozin propanediol, dapagliflozin propanediol+sitagliptin phosphate, dapansutrile, DBI-500, DC-291407, DCR-CLD, DCR-LIV2, DD-01, DD-02, DD-03, DD-15, denatonium acetate monohydrate, dendritic cell-based microsphere vaccine, denifanstat, Des-1 inhibitor, DFV-890, DiabeCell, Diamyd, DLA bispecific antibody program, DLBS-3233, DM-1050, D-methyldopa, DNX-314, dorzagliatin, DR-10624, DT-109, dual agonist combination 1 therapy, dual agonist combination 3, dual GLP1/glucagon receptor agonists, dual PPAR-alpha/gamma agonists, dual PPAR-gamma/delta modulators, Duglow, dulaglutide, DWC-202001, DWC-202002, DWC-202213, DWJ-1525, DWJ-1563, DWP-457, DWRX-5003, EA-3571, EBX-102, ECC-0509, ECC-4703, ECC-5004, ecnoglutide, edaravone, efinopegdutide, eflornithine, efpeglenatide, efruxifermin, elafibranor, elebsiran, elobixibat, EM-101, Enterome, EMI-137, empagliflozin, empagliflozin+linagliptin, empagliflozin+linagliptin+metformin XR, empagliflozin+metformin, empagliflozin+metformin extended-release, enavogliflozin, ENB-106, ENB-110e, ENC-201, encapsulated Melligen cell therapy, ENERGI-F702, engineered regulatory T cell therapy, ENN-0403, ENT-001, epeleuton, EPGN-696, Epi-13, ERp44-adiponectin interactions modulators, ertugliflozin, ertugliflozin+metformin, ertugliflozin+sitagliptin, ervogastat, ETX-291, ETX-312, evexomostat, evogliptin, evogliptin+dalpagliflozin+metformin, evogliptin+metformin XR, Exd-391209, exenatide, exendin-4-Fc fusion protein, exendin-4-human serum albumin fusion protein, EXG-34217, exosome based therapeutic, extended release glipizide, EYP-002, ezurpimtrostat, F-573, farnesoid X receptor agonists, fazirsiran sodium, FB-102, FB-1603, FB-1807, FBPase inhibitors, fenofibrate+atorvastatin, fezagepras, FFAR4 agonists, FGF21 gene therapy, FIA-586, FIB-918, FIB-992, firsocostat, fluasterone, fluorofenidone, FM-101, foralumab, foscenvivint, fotagliptin benzoate, FOXO1 inhibitor program, FP10.47, fresolimumab, frexalimab, FRTX-02, FSI-965, FT-4101, Fuzheng Huayu capsule, FXR agonists, FXR-314, G3PP activator program, G-49, GABA+antigen based therapy, GAD-65, Gal-300, galectin-3 inhibitors, gallium (68Ga) rofapitide tetraxetan, GalNAc-conjugated siRNA therapeutic, GARV-AAV2-A20, GB-7001, G2GBio, GDD-3898, GDNF, gemigliptin+dapagliflozin, gemigliptin+metformin HCl, gemigliptin+rosuvastatin, gemigliptin tartaric acid, GEN-1503PR, GF-1002, GH-509, GI-210, GKAC, GL-0034, glargine biosimilar, glibenclamide, gliclazide, glimepiride, glimepiride+extended-release metformin hydrochloride, glimepiride+metformin, glimepiride+vildagliptin+metformin, GLP-1 analog-COL3A1 fusion protein, GLP-1 analogs, GLP-1 receptor agonist, GLP-1/exendin-4-Fc fusion proteins, GLP-1/GIP receptor agonist, GLP-1-Fc-PYY, glucagon-like peptide-1 analog, glucose responsive smart basal insulin, glutazumab, GLY-200, GLY-POL, GM-60106, GM-90194, GMA-106, GMA-107, GMA-1OX, GMA-10Y, GNF-2133, GNS-3595, golexanolone, golimumab, gosogliptin, GPR39 antagonists, GPX-002, GPX-003, GR-012, GR-018, GR-019, GRI-0124, GS-300, GS-834356, GSBR-1290, GST-HG-151, GTX-011, GV-101, GX-G6, GXHPC-1, GXIPC-1, GZR-101, GZR-18, GZR-4, HB-1085, HB-601, HCL-001, HD-1916, HD-7671, HDM-1002, HDNO-1605, HEC-192334, HEC-44616, HEC-88473, HEC-96719, HepaStem, heterologous liver cell therapy, HGR-4113, HJ-178, HJC-0416, HK-1, HK-660S, HL-012MA, HL-08, HLA-DQ2/DQ8 inhibitors, HM-12460A, HM-15136+efpeglenatide, HM-15211, HM-15275, HNF4A mRNA therapeutics, HP-515, HPD-001, HPG-1860, HPG-5119, HPG-7233, HPN-01, HPN-F01, HPP-3033, HR-17031, HR-19042, HR-20033, HRS-7535, HRS-9531, HRX-0215, HRX-0701, HS-10356, HS-10501, HS-20004, HS-20094, HSD17B13 inhibitors, HSD17b13i, HSK-31679, HSK-34890, HT-201, HT-202, HTL-30023, HTPEP-002, HU-6, human albumin, human amniotic exosomes, human FGF-1, human insulin biosimilar, human plasma gelsolin, human pluripotent embryonic stem cell-derived pancreatic islet cells, human pluripotent stem cells derived allogenic hepatocytes, humanin analogs, HX-100101-1, HX-1171, HY-209, HYBR-011, hydrogel-exenatide, hydronidone, hydroxyl dendrimer-alendronate conjugate, hymecromone, hypoglycemic agents, HZ-010, HZ-012, i2o-107, i2o-110, i2o-120, ianalumab, IAPP inhibitor, ibrigampar, Ibutamoren, icomidocholic acid, icosabutate, icovamenib, ID-110521156, ID-11903, ID-119050134, IDG-16177, IDL-2965, IFNalpha kinoide, Ii-key/MHC class II epitope hybrid peptide immunomodulator peptide vaccines, IL-1512, IL-2 fusion protein, ILV-001, IM-102, IMC-S118AI, IMCY-0098, imeglimin, IMG-1, IMM-124-E, IMM-H014, ImmTOR+PDC-E2, IMT-001, IN-A004, IN-A010, INI-822, INS-068, insulin lispro, insparin, insulin, insulin aspart, insulin degludec, insulin degludec+insulin aspart, insulin degludec+liraglutide, insulin degrading enzyme inhibitors, insulin efsitora alfa, insulin glargine, insulin glargine+lixisenatide, insulin icodec, insulin lispro, insulin lispro+exenatide, insulin lispro+pramlintide, insulin mouth rinse, insulin tregopil, insulin-producing stem-islet beta cell hybrids, Insuman Implantable, INT-787, interferon alfa-2a follow-on biologic, interferon alfa-n1, interferon gamma follow-on biologic, INV-002, INVA-8001, invopressin, IOA-289, ION-224, ION-769357, IONIS-GCGRRx, IOT-022, IPG-008, IPN-60250, ipragliflozin, ipragliflozin+sitagliptin, iPSC cell therapy, iPSC-derived pancreatic islet cell therapy, IRE1alpha inhibitors, iscalimab, ISM-001, ITCA-1601, iTOL-101, iTOL-102, ITV-1, IVB-001, ivonescimab, iXB-401, J2H-1702, JAG-301, JMT-202, JP-2266, JT-003, JTT-251, JTT-662, JW-0201, K-757, K-833, kamuvudines, KBL-982, KBLP-004, KBP-336, KD-4002, KD-4004, KGYY-15, KH-629, KH-805, KH-806, KN-056, KQ-791, KSHB-005, KT-A112, KT-A522, KT-A832, Kv1.3 ion channel mAbs, KY-41111, KYLO-0603, L-47, LABP-111, LABthera-006, ladarixin, LAE-103, LAE-104, LAE-105, LAE-106, laflunimus, lanifibranor, lapretolimod, larsucosterol, LB-P7, LB-P8, LBS-009, LC-542019, LEM-S402, leronlimab, leucine+metformin+sildenafil, LG-203003, L-glutamine, LH-1801, licogliflozin bis(prolinate), linagliptin, linagliptin+dapagliflozin+metformin hydrochloride, linagliptin+metformin hydrochloride, linagliptin+metformin XR, linerixibat, liraglutide, liver disease/non-alcoholic steatohepatitis therapy, lixisenatide, LM-011, L-methyldopa, LN-3118, LNP-CDP mRNA therapy, lobeglitazone+metformin, lobeglitazone+sitagliptin, LP-342, LPCN-1148, LPS-001, LPXT-007, LR-1, LR-19018, LR-19131, LR-20022, LRH-1 modulators, LUNAR-TKD, luseogliflozin hydrate, LXR inverse agonists, LY-3493269, LY-3522348, LY-3532226, LY-3537021, LY-3549492, LY-3849891, LY-3885125, LY-3938577, LY900027, LYS-006, M-008, M-43, maralixibat chloride, maraviroc, masoprocol, mazdutide, MB-204, MB-N-008, MB-X01Y03, mCLC-846, mecasermin, memantine derivatives, MET-409, metformin, metformin+glibenclamide, metformin glycinate, metformin hydrochloride, metformin XR+valsartan+atorvastatin, MFC-0101, MFC-0102, MHS-552, mibavademab, microbiome-based live bacterial probiotic, microparticle vaccine, miglitol, miR-132 antisense oligonucleotide inhibitor, miricorilant, MI-S4, mitiglinide, mitiglinide+metformin, mitiglinide+voglibose, mitiperstat, mitochondrial uncouplers, MK-1092, MK-3655, MK-6204, MK-7480, MKP-10241, MLX-0800, MLX-5000, MLX-7000, MNO-863, monlunabant, MORF-627, mosedipimod, MPC-300-IV, MRG-229, mRNA-6981, mRNA-LNPs expressing EGF and HGF, MT-1002, MT-2002, MT-2004, MTX-101, MTX-463, MTX-474, MWN-101, MWN-102, myonectin, NA-941, naltrexone, namodenoson, nanofitins, nateglinide, nateglinide+metformin hydrochloride, NC-101, NDC-0009, necroptosis inhibitors, nerigliatin, netakimab, netazepide, NeuLiv, neuropeptide Y2-receptor agonists, NEXI-005, next generation peptidomimetic and tissue transglutaminase inhibitors, next generation smart glucose-responsive insulin, NGM-395, NI-203, nibrozetone, nicotinamide N-methyltransferase inhibitor, nimotuzumab, ninerafaxstat trihydrochloride monohydrate, NIPEP-CARE, nisotirostide, nitazoxanide, NJA-005, NKTT-320, NLRP3 gene therapy, NMX-2, NN-1471, NN-1535, NN-1845, NN-6177, NN-6561, NN-6581, NN-6582, NN-9041, NN-9541, NNC-0113-6856, NNC0113-6860, NNC0113-6861, NNC0113-6891, NNC-0194-0499, NNC-0472-0147, NNC0480-0389, NNC-0519-0130, NNC-0650-0013, NNC6022-0001, NNC-965, non-alcoholic fatty liver disease agent, norucholic acid, NovDB2, NovFS, NOX inhibitors, NOX4 inhibitors, NP-011, NTCP inhibitor, nucleoside-modified TERT mRNA, NV-422, NV-556, NX-9001, 0-01, OATD-01, obefazimod, obeticholic acid, obeticholic acid magnesium, OBM-P01, odevixibat, OGB-21501, OGB-21502, olmesartan+dapagliflozin, oltipraz, OLX-701, OLX-702A, omarigliptin, OPC-163493, OPT-101, ORBCEL-C, orforglipron, ORMD-0801, ORMD-0801+ORMD-0901, ORMD-0901, OsrHSA, otelixizumab, Oxy-210, P-11, pancreatic beta cell modulators, PanINSULA, PAR2 inhibitor pepducins, PB-718, PBI-4547, PEC-Direct, PEC-EnCap, pegapamodutide, pegargiminase, pegbelfermin, PEG-exenatide, peginterferon alfa-2a, peginterferon alfa-2b, PEG-loxenatide, pegozafermin, PEGylated exenatide, pemafibrate, pemafibrate+tofogliflozin, pemvidutide, Peptide, peptide-loaded nanoparticles, petrelintide, PF-06835919, PF-06954522, PF-07853578, PGC-fatylated terlipressin, PGN-0B2, PHIN-214, PHP-303, piclidenoson, pioglitazone, pioglitazone+extended-release metformin, pioglitazone+glimepiride, pioglitazone+metformin, pioglitazone+teneligliptin, PKX-001, PLN-1474, PNPLA3 inhibitor, PPAR-gamma agonists, pramlintide+exenatide, pramlintide+human insulin, preimplantation factor, PRIM-DJ2727, PRL-002, PRO-20, ProAgio, proglumide, Prolastin, proline henggliflozin+retagliptin phosphate+metformin hydrochloride, ProTrans, prusogliptin, PRV-101, PS-1, psilocybin, PSTvl, PT-001, PT-002, PTG-007, PTG-020, PTPN1 siRNA therapy, PTUPB, PVT-101, PXL-065, PXL-770, PXS-5382, pyrazole carboxamide analogs, PZH-2109, Qaialdo, QBT-002, QLR-12018, QPCT inhibitor, QRB-001, quisovalimab, R-0737072, R-2487, radiolabelled FAPI-46, rapirosiran, RAY-001, RAY-002, RAY-1225, RBP4 antagonist, RCYM-001, rE-4, recombinant angiotensin converting enzyme 2 (ACE-2) gene therapy/AAV vector, recombinant CTRP12 protein, remogliflozin etabonate, remogliflozin etabonate+teneligliptin, remogliflozin etabonate+vildagliptin, remogliflozin etabonate+vildagliptin+metformin, rencofilstat, REP-2139, repaglinide, repaglinide+metformin, repaglinide+metformin hydrochloride, RES-010, ReS39-J program, resmetirom, retagliptin, retatrutide, RG-125, rGDF11, RGT-028, RGT-075, RhuDex, rifaquizinone, rifaximin, rilparencel, ritlecitinib, RJ-4287, RJVA-001, RMD-1201, rosiglitazone, rosiglitazone+glimepiride, rosiglitazone maleate+metformin hydrochloride, rosiglitazone sodium, rosuvastatin+telmisartan+amlodipine, RP-005, RSVI-301, RT-200, RTX-001, RTX-T1D, Ryzodeg, RZ-629, SAB-142, SAL-0112, SAL-067, SAMiRNA program, SAMiRNA-AREG, santamarin derivatives, saRNA therapeutics, saroglitazar magnesium, saxagliptin, saxagliptin+dapagliflozin, saxagliptin+dapagliflozin+metformin hydrochloride, saxagliptin+extended-release metformin, saxagliptin+metformin, SBI-102, SBP-302, SBT-11-5301, SC-451, SCO-094, SCO-116, SCO-267, SCT-5-27, second generation chitotriosidase 1 inhibitor, second generation lecinoxoids, second generation naltrexone analog, seladelpar lysine, selatogrel, selvigaltin, semaglutide, SER-140, serelaxin, setanaxib, sevelamer hydrochloride, SFA-001, SFX-01, SGM-1019, SH-2442, SHC-023, SHC-028, Shiloah-1000, shortened GLP-1, SHR-2042, SHR-3167, SHR-3824, SIG-002, siplizumab, siRNA therapy, SIRT6 gene therapy, SIRT6 inhibitors, Sirtuin6 protein regulators, sitagliptin, sitagliptin+extended-release metformin, sitagliptin+glimepiride+metformin hydrochloride ER, sitagliptin+metformin, sitagliptin+metformin+pioglitazone, sitagliptin+simvastatin, sitagliptin fenilalanil hydrochloride, SK1-I, SL-100, SN-401, SN-406, SNP-610, SNP-630, soluble guanylate cyclase stimulator, sotagliflozin, SPL-891, SPN-0103-009, SPP-004, SQZ-TAC-T1D, SR-03, SR-040/041/042, SR-044, SRI-37330, SRT-015, ST-003, STM-003, STP-707, supalutai, survodutide, SVP insulin+SVP rapamycin, SVT-201, SY-004, SY-008, SY-009, SYHA-1805, SYN-020, SZN-043, T-1123, T-1123, T1D-ADV210, T2D-PT201, tacrolimus, targeted gene therapy, TB-001, TB59-2, TB-840, TB-D-004C, T-cell expressed protein, TCM-800B, TCR-TREGs, TDI-01, TE-8105, technetium Tc 99m tilmanocept, telazorlimab, temelimab, teneligliptin+metformin, teneligliptin hydrobromide, teneligliptin hydrobromide+canagliflozin hydrate, teplizumab, TERN-101, TERN-501, tesamorelin, testosterone prodrug, testosterone undecanoate, TG-68, TH-104, THDBH-101, THDBH-110/THDBH-111, THDBH-120/THDBH-121, thioacrylamide compounds, thyroid hormone receptor beta-selective agonists, tianagliflozin, TID-PT101, tilpisertib, timolumab, tipelukast, tirzepatide, tissue-specific immunomodulating bispecific antibodies, TIX-100, TJ-103, TJC-0316, TLC-065, TLC-1235, TLC-2716, TLC-3595, TLC-6740, TLY-004, TLY-012, tofogliflozin, TOL-3021, ToleraCell-001, ToleraCell-002, tolimidone, TP-352, TQ-05510, TQA-3526, TQ-F3083, Treg modulator therapy, trelagliptin succinate, TreXTAM, tricyclic pyrone compounds, TS-20004, TSG-03-117, TSL-0319, TT-01025, TTP-273, TTP-RA, TU-5113, tulinercept, TXR-611, TXR-612, TY-705, UBT-251, UI-068, ularitide, Ulinastatin, umbilical cord blood-derived mesenchymal stem cell therapy, umbilical cord-derived mesenchymal stem cells, UN-03, Uni-E4-Fc, UP-421, ursodeoxycholic acid, ursolic acid, UTAA-09, V-411, valsartan+celecoxib, VAR 200-03, VB-201, VB-601, VCT-220, VCTX-210, VCTX-212, VE-5708, VE-5773, vesicular monoamine transporter 2 antagonists, vidofludimus, vildagliptin, vildagliptin+metformin, vildagliptin+pioglitazone, vildagliptin+pioglitazone hydrochloride, vildagliptin SR+metformin SR, visepegenatide, VK-0612, VK-2735, VK-2735, oral, VK-2809, VNA-438, VN-B101, voglibose, voglibose+metformin, volagidemab, volixibat potassium ethanolate hydrate, vonafexor, vonifimod, VS-105, VUM-02, vutiglabridin, VX-264, VX-880, wanpagliflozin, WCDD-301, WS-012, WXSH-0038, WXSH-0078, WXSH-0213, XEN-103, XEN-D0501, xenin-fused peptides, XFB-19, XP-3924, XTYW-003, XW-013, XW-014, XW-015, XZP-5610, XZP-5695, XZP-6019, YA-6060, YD02-2022, YFQLXB-UCO1, YG-1699, YH-2000, YH-25724, YH-40863, YHC-1102, YHC-1108, YHC-1131, YJH-0425, yogliptin, YW-1128, YYC-405, ZED-1227, zelasudil, zetomipzomib, zevaquenabant, ZG-0588, zibotentan, ziftomenib, Zituvio, ZMC-001, ZSP-0678, ZSP-1601, ZSYM-008, ZT-002, ZT-003, ZT-01, ZX-2010, ZX-2020, ZX-2021, or bi-specific antibodies targeting one or more targets referenced herein.

In some embodiments a compound of the disclosure provided herein, or pharmaceutically acceptable salt thereof, is administered with one or more therapeutic agents selected from a PPARδ inhibitor, IRAK4 inhibitor, TPL2 inhibitor, α4β7 inhibitor, BTLA agonist, PD1 agonist, or an FXR agonist.

In some embodiments a compound of the disclosure provided herein, or pharmaceutically acceptable salt thereof, is administered with one or more therapeutic agents selected from seladelpar, edecesertib, tilpisertib fosmecarbil, GS-1427, GS-0272, GS-0151, GS-8670, or cilofexor.

### Pharmaceutical Compositions, Dosage Form, and Modes of Administration

A FXR agonist, seladelpar, or optional additional therapeutic agent provided herein are usually administered in the form of pharmaceutical compositions. Thus, provided herein are also pharmaceutical compositions that contain a FXR agonist and seladelpar or a pharmaceutically acceptable salt, tautomer, stereoisomer, mixture of stereoisomers, prodrug, or deuterated analog thereof and one or more pharmaceutically acceptable vehicles selected from carriers, adjuvants, and excipients. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

The pharmaceutical compositions may be administered in either single or multiple doses. The pharmaceutical composition may be administered by various methods including, for example, rectal, buccal, intranasal, and transdermal routes. In certain embodiments, the pharmaceutical composition may be administered by intra-arterial injection, intravenously, intraperitoneally, parenterally, intramuscularly, subcutaneously, orally, topically, or as an inhalant.

One mode for administration may be oral. Administration may be via, for example, capsule or enteric coated tablets. In making the pharmaceutical compositions that include at least one compound described herein or a pharmaceutically acceptable salt, tautomer, stereoisomer, mixture of stereoisomers, prodrug, or deuterated analog thereof, the active ingredient is usually diluted by an excipient and/or enclosed within such a carrier that can be in the form of a capsule, sachet, paper, or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

In some embodiments, a FXR agonist, seladelpar, or optional additional therapeutic agent may be provided as a capsule or tablet for oral administration.

The pharmaceutical composition within the capsule may include about 3 mg to about 100 mg, about 3 mg to about 50 mg, about 3 mg to about 20 mg, about 5 mg to about 20 mg, about 3 mg to 15 mg, about 5 mg to 15 mg, or about 7 mg to 12 mg equivalent amount of seladelpar. In some embodiments, the pharmaceutical composition may include about 10 mg, 20 mg, 30 mg, 40 mg, 50 mg, or 100 mg equivalent amount of seladelpar. In some embodiments, the seladelpar may be provided as lysine (e.g., L-lysine) salts dihydrate. The pharmaceutical composition may include about 14.1 mg of seladelpar lysine dihydrate.

The pharmaceutical composition may include about 3 mg to about 200 mg, about 10 mg to about 200 mg, about 10 mg to about 150 mg, about 10 mg to about 100 mg, about 20 mg to 100 mg, about 20 mg to 100 mg, or about 20 mg to 50 mg equivalent amount of a FXR agonist. In some embodiments, the pharmaceutical composition may include about 10 mg, about 20 mg, about 30 mg, about 40 mg, about 50 mg, about 60 mg, about 70 mg, about 80 mg, about 90 mg, about 100 mg, about 150 mg, or about 200 mg equivalent amount of a FXR agonist, such as,

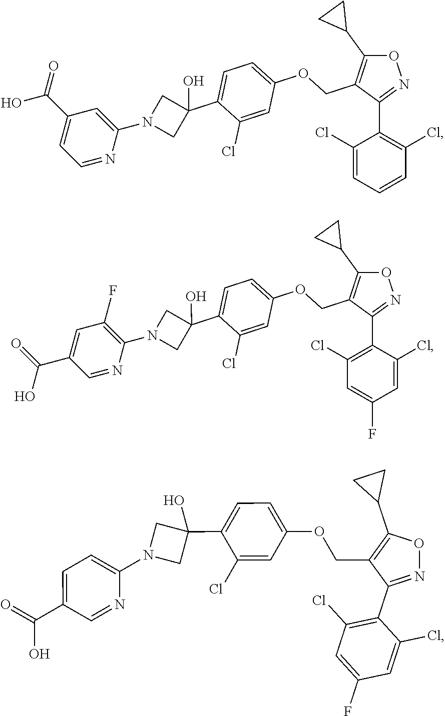

F, or a pharmaceutically acceptable salt thereof.

In some embodiments, the FXR agonist, such as

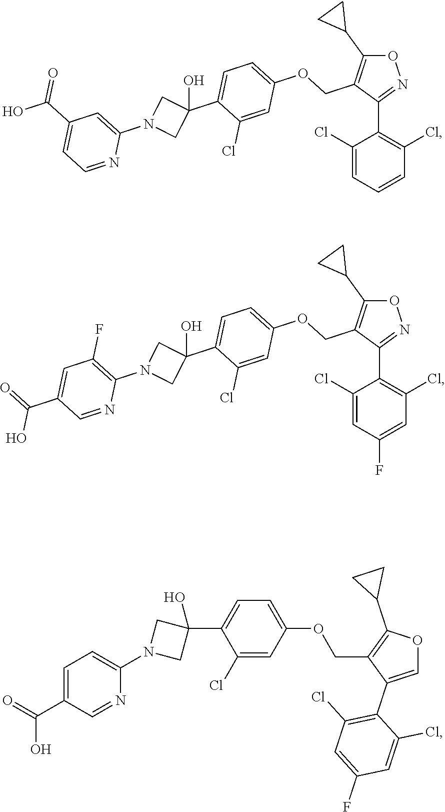 [SMILES: O=C(O)c1ccnc(N2CC(O)(c3ccc(OCc4c(-c5c(Cl)cccc5Cl)noc4C4CC4)cc3Cl)C2)c1 ; O=C(O)c1cnc(N2CC(O)(c3ccc(OCc4c(-c5c(Cl)cc(F)cc5Cl)noc4C4CC4)cc3Cl)C2)c(F)c1 ; O=C(O)c1ccc(N2CC(O)(c3ccc(OCc4c(-c5c(Cl)cc(F)cc5Cl)coc4C4CC4)cc3Cl)C2)nc1]

may be provided as zwitterion, tromethamine salt, or p-TSA salt.

The pharmaceutical composition may include one or more of the following excipients: butylated hydroxytoluene, colloidal silicon dioxide, croscarmellose sodium, magnesium stearate, mannitol, and microcrystalline cellulose. In some embodiments, the pharmaceutical composition includes butylated hydroxytoluene, colloidal silicon dioxide, croscarmellose sodium, magnesium stearate, mannitol, and microcrystalline cellulose.

The pharmaceutical composition may be contained within hard gelatin capsule shells. The capsule shells may include gelatin, titanium dioxide, black iron oxide, yellow iron oxide, red iron oxide and/or colorants.

The compositions that include at least one compound described herein or a pharmaceutically acceptable salt, tautomer, stereoisomer, mixture of stereoisomers, prodrug, or deuterated analog thereof can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods disclosed herein employ transdermal delivery devices (“patches”). Such transdermal patches may be used to provide continuous or discontinuous infusion of the compounds described herein in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein or a pharmaceutically acceptable salt, tautomer, stereoisomer, mixture of stereoisomers, prodrug, or deuterated analog thereof. When referring to these preformulation compositions as homogeneous, the active ingredient may be dispersed evenly throughout the composition so that the composition may be readily subdivided into equally effective unit dosage forms such as tablets, pills, and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Compositions for inhalation or insufflation may include solutions and suspensions in pharmaceutically acceptable, aqueous, or organic solvents, or mixtures thereof, and powders. The liquid or solid compositions may contain suitable pharmaceutically acceptable excipients as described herein. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

### Kits

Provided herein are also kits that include a compound of the disclosure, or a pharmaceutically acceptable salt, tautomer, stereoisomer, mixture of stereoisomers, prodrug, or deuterated analog thereof, and suitable packaging. In one embodiment, a kit further includes instructions for use. In one aspect, a kit includes a compound of the disclosure, or a pharmaceutically acceptable salt, tautomer, stereoisomer, mixture of stereoisomers, prodrug, or deuterated analog thereof, and a label and/or instructions for use of the compounds in the treatment of the indications, including the diseases or conditions, described herein.

Provided herein are also articles of manufacture that include a compound described herein or a pharmaceutically acceptable salt, tautomer, stereoisomer, mixture of stereoisomers, prodrug, or deuterated analog thereof in a suitable container. The container may be a vial, jar, ampoule, preloaded syringe, and intravenous bag.

### Dosing

The specific dose level of a compound of the present application (e.g., a FXR agonist and seladelpar) for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

The daily dosage may also be described as a total amount of a compound (e.g., a FXR agonist and seladelpar) described herein administered per dose or per day. Daily dosage of seladelpar may be between about 1 mg and 4,000 mg, between about 2,000 to 4,000 mg/day, between about 1 to 2,000 mg/day, between about 1 to 1,000 mg/day, between about 10 to 500 mg/day, between about 20 to 500 mg/day, between about 50 to 300 mg/day, between about 75 to 200 mg/day, or between about 5 to 150 mg/day.

When administered orally, the total daily dosage of a compound (e.g., a FXR agonist and seladelpar) for a human subject may be between 1 mg and 1,000 mg, between about 1,000-2,000 mg/day, between about 10-500 mg/day, between about 50-300 mg/day, between about 75-200 mg/day, or between about 100-150 mg/day.

In some embodiments, a FXR agonist, seladelpar, or optional additional therapeutic agent is administered in an amount of about 0.1 mg/day to about 1200 mg/day. In some embodiments, a FXR agonist, seladelpar, or optional additional therapeutic agent is administered in an amount of 1 mg/day to about 100 mg/day, about 10 mg/day to about 1200 mg/day, about 10 mg/day to about 100 mg/day, about 100 mg/day to about 1200 mg/day, about 400 mg/day to about 1200 mg/day, about 600 mg/day to about 1200 mg/day, about 400 mg/day to about 800 mg/day, or about 600 mg/day to about 800 mg/day. In some embodiments, methods disclosed herein comprise the administration of 0.1 mg/day, 0.5 mg/day, 1 mg/day, 10 mg/day, 15 mg/day, 20 mg/day, 30 mg/day, 40 mg/day, 45 mg/day, 50 mg/day, 60 mg/day, 75 mg/day, 100 mg/day, 125 mg/day, 150 mg/day, 25 200 mg/day, 250 mg/day, 300 mg/day, 400 mg/day, 600 mg/day or 800 mg/day of a FXR agonist, seladelpar, or optional additional therapeutic agent subject in need thereof.

In some embodiments, the total daily dose of a FXR agonist, seladelpar, or optional additional therapeutic agent is selected from about 5 mg, about 10 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 5 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, 10 about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, about 2000 mg, about 2050 mg, about 2100 mg, about 2150 mg, about 2200 mg, about 2250 mg, about 2300 mg, about 2350 mg, about 2400 mg, about 2450 mg, about 2500 mg, about 2550 mg, about 2600 mg, about 2650 mg, about 2700 mg, about 2750 mg, about 2800 mg, about 2850 mg, about 2900 mg, about 2950 mg, or about 3000 mg.

In some embodiments, the total daily dose of a FXR agonist, seladelpar, or optional additional therapeutic agent is independently between about 5 mg to about 3000 mg, between about 5 mg to about 1000 mg, between about 5 mg to about 500 mg, between about 5 mg to about 100 mg, between about 10 mg to about 3000 mg, between about 10 mg to about 2000 mg, between about 10 mg to about 1000 mg, between about 20 mg to 30 about 1000 mg, between about 30 mg to about 1000 mg, between about 30 mg to about 750 mg, between about 30 mg to about 500 mg, between about 30 mg to about 250 mg, between about 30 mg to about 100 mg, between about 50 mg to about 500 mg, or between about 50 mg to about 100 mg.

The compounds of the present application or the compositions thereof may be administered once, twice, three, or four times daily, using any suitable mode described above. Also, administration or treatment with the compounds may be continued for a number of days; for example, commonly treatment would continue for at least 7 days, 14 days, or 28 days, for one cycle of treatment. Treatment cycles are well known in cancer chemotherapy, and are frequently alternated with resting periods of about 1 to 28 days, commonly about 7 days or about 14 days, between cycles. The treatment cycles, in other embodiments, may also be continuous.

In a particular embodiment, the method comprises administering to the subject an initial daily dose of about 1 to 800 mg of a compound described herein and increasing the dose by increments until clinical efficacy is achieved. Increments of about 5, 10, 25, 50, or 100 mg can be used to increase the dose. The dosage can be increased daily, every other day, twice per week, or once per week.

In some embodiments, seladelpar or pharmaceutically acceptable salt thereof may be administered in an amount equivalent to 10 mg/day of seladelpar.

### EMBODIMENTS

Embodiment 1. A method of treating and/or preventing a liver disease in a patient in need thereof, the method comprising administering a therapeutically effective amount of a FXR agonist or a pharmaceutically acceptable salt thereof and a therapeutically effective amount of seladelpar or a pharmaceutically acceptable salt thereof to the patient.

Embodiment 2. The method of Embodiment 1, wherein the FXR agonist is obeticholic acid (OCA), GW4064, ASC-42, PX-104, TERN 101, cilofexor, GS-8670, tropifexor, vonafexor, EDP-305, MET-409, or a pharmaceutically acceptable salt thereof.

Embodiment 3. The method of Embodiment 1, wherein the FXR agonist is:

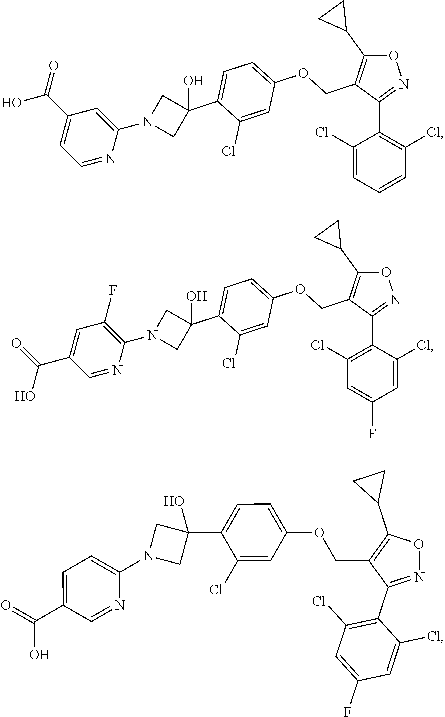

or a pharmaceutically acceptable salt thereof.

Embodiment 4. The method of any one of Embodiments 1-3, wherein the patient is administered seladelpar lysine.

Embodiment 5. The method of any one of Embodiments 1-3, wherein the patient is administered seladelpar L-lysine.

Embodiment 6. The method of any one of Embodiments 1-3, wherein the patient is administered seladelpar L-lysine dihydrate.

Embodiment 7. The method of any one of Embodiments 1-6, wherein the therapeutically effective amount of seladelpar or the pharmaceutically acceptable salt thereof is equivalent to about 10 mg/day of seladelpar.

Embodiment 8. The method of any one of Embodiments 1-7, wherein the liver disease is metabolic dysfunction-associated steatotic liver disease (MASLD), primary biliary cholangitis (PBC), or primary sclerosing cholangitis (PSC).

Embodiment 9. The method of any one of Embodiments 1-8, wherein the patient has been previously administered the FXR agonist and the patient was associated with pruritus.

Embodiment 10. The method of Embodiment 9, wherein the pruritus is FXR-induced.

Embodiment 11. The method of Embodiment 9 or 10, wherein the pruritus is due to the patient being administered the FXR agonist.

Embodiment 12. The method of any one of Embodiments 1-11, wherein the FXR agonist and seladelpar are administered together.

Embodiment 13. The method of any one of Embodiments 1-11, wherein the FXR agonist and seladelpar are administered separately.

Embodiment 14. The method of any one of Embodiments 1-11, further comprising administering UDCA to the patient.

Embodiment 15. A method of treating and/or preventing primary biliary cholangitis (PBC) in a patient in need thereof, the method comprising administering a therapeutically effective amount of cilofexor or a pharmaceutically acceptable salt thereof and a therapeutically effective amount of seladelpar or a pharmaceutically acceptable salt thereof to the patient.

Embodiment 16. The method of Embodiment 15, wherein the patient is administered seladelpar lysine.

Embodiment 17. The method of Embodiment 15 or 16, wherein the patient is administered seladelpar L-lysine.

Embodiment 18. The method of any one of Embodiments 15-17, wherein the patient is administered seladelpar L-lysine dihydrate.

Embodiment 19. The method of any one of Embodiments 15-18, wherein the therapeutically effective amount of seladelpar or the pharmaceutically acceptable salt thereof is equivalent to about 10 mg/day of seladelpar.

Embodiment 20. The method of any one of Embodiments 15-19, wherein the FXR agonist and seladelpar are administered together.

Embodiment 21. The method of any one of Embodiments 15-19, wherein the FXR agonist and seladelpar are administered separately.

Embodiment 22. A method of treating pruritus comprising administering to a patient in need thereof a therapeutically effective amount of seladelpar or a pharmaceutically acceptable salt thereof.

Embodiment 23. The method of Embodiment 22, wherein the pruritus is Farnesoid X Receptor (FXR)-induced pruritus.

Embodiment 24. The method of Embodiment 22 or 23, wherein the patient is suffering from a liver disease.

Embodiment 25. The method of Embodiment 24, wherein the liver disease is selected from: metabolic dysfunction-associated steatohepatitis (MASH), formally non-alcoholic steatohepatitis (NASH), primary sclerosing cholangitis (PSC), primary biliary cirrhosis (PBC), autoimmune hepatitis, alcoholic steatohepatitis (ASH), liver fibrosis, liver cirrhosis, liver steatosis (fatty liver disease), liver ischemia (liver injury in Cortellis), metabolic dysfunction-associated steatotic liver disease (MASLD), and progressive familiar intrahepatic cholestasis (PFIC).

Embodiment 26. The method of any one of Embodiments 22-24, wherein the patient is suffering from metabolic dysfunction-associated steatotic liver disease (MASLD), primary biliary cholangitis (PBC), or primary sclerosing cholangitis (PSC).

Embodiment 27. The method of Embodiment 23, wherein the FXR-induced pruritus is due to the patient being administered an FXR agonist or due to a bile acid.

Embodiment 28. The method of Embodiment 27, wherein the FXR agonist is obeticholic acid (OCA), GW4064, ASC-42, PX-104, TERN 101, cilofexor, GS-8670, tropifexor, vonafexor, EDP-305, MET-409, or a pharmaceutically acceptable salt thereof.

Embodiment 29. The method of Embodiment 27, wherein the FXR agonist is:

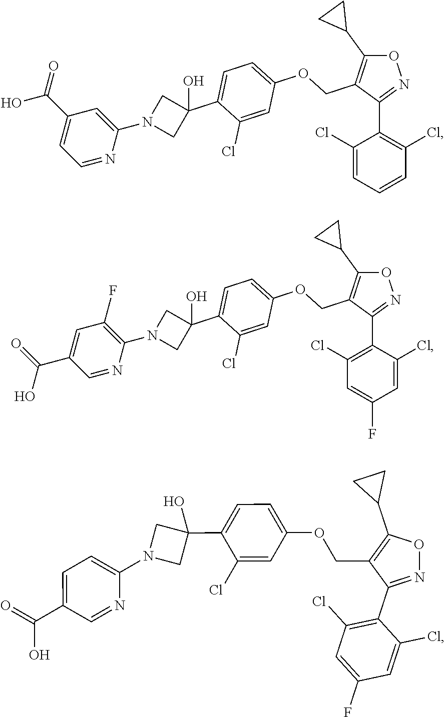

or a pharmaceutically acceptable salt thereof.

Embodiment 30. The method of Embodiment 23, wherein the FXR-induced pruritus is associated with elevated serum IL-31 levels due to increased serum bile acid levels.

Embodiment 31. A method of improving cholestasis in a patient in need thereof, the method comprising administering a therapeutically effective amount of a FXR agonist or a pharmaceutically acceptable salt thereof and a therapeutically effective amount of seladelpar or a pharmaceutically acceptable salt thereof to the patient.

Embodiment 32. A method of reducing bile acid in a patient in need thereof, the method comprising administering a therapeutically effective amount of a FXR agonist or a pharmaceutically acceptable salt thereof and a therapeutically effective amount of seladelpar or a pharmaceutically acceptable salt thereof to the patient.

Embodiment 33. A method of reducing at least one of cholestatic injury, inflammation, or liver fibrosis in a patient in need thereof, the method comprising administering a therapeutically effective amount of a FXR agonist or a pharmaceutically acceptable salt thereof and a therapeutically effective amount of seladelpar or a pharmaceutically acceptable salt thereof to the patient.

Embodiment 34. The method of any one of Embodiments 31-33, wherein the FXR agonist is obeticholic acid (OCA), GW4064, ASC-42, PX-104, TERN 101, cilofexor, GS-8670, tropifexor, vonafexor, EDP-305, MET-409, or a pharmaceutically acceptable salt thereof.

Embodiment 35. The method of any one of Embodiments 31-33, wherein the FXR agonist is:

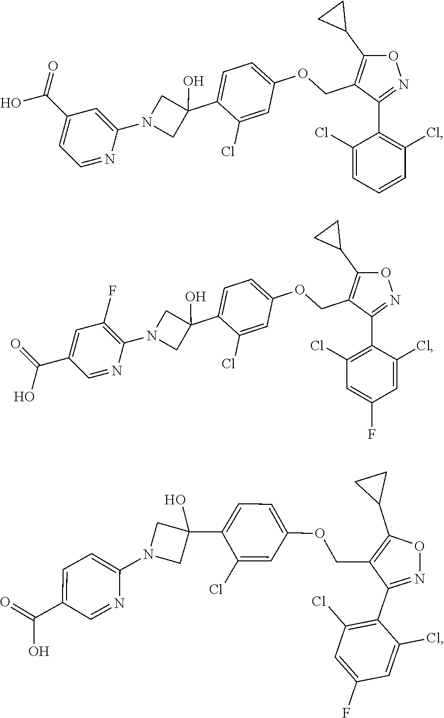

or a pharmaceutically acceptable salt thereof.

Embodiment 36. The method of any one of Embodiments 31-35, wherein the patient is administered seladelpar lysine.

Embodiment 37. The method of any one of Embodiments 31-35, wherein the patient is administered seladelpar L-lysine.

Embodiment 38. The method of any one of Embodiments 31-35, wherein the patient is administered seladelpar L-lysine dihydrate.

Embodiment 39. The method of any one of Embodiments 31-38, wherein the therapeutically effective amount of seladelpar or the pharmaceutically acceptable salt thereof is equivalent to about 10 mg/day of seladelpar.

Embodiment 40. The method of any one of Embodiments 31-39, wherein the patient is suffering from a liver disease.

Embodiment 41. The method of any one of Embodiments 40, wherein the patient is suffering from metabolic dysfunction-associated steatotic liver disease (MASLD), primary biliary cholangitis (PBC), or primary sclerosing cholangitis (PSC).

Embodiment 42. The method of any one of Embodiments 31-41, wherein the FXR agonist and seladelpar are administered together.

Embodiment 43. The method of any one of Embodiments 31-41, wherein the FXR agonist and seladelpar are administered separately.

Embodiment 44. A composition comprising a therapeutically effective amount of an FXR agonist and a therapeutically effective amount of seladelpar or a pharmaceutically acceptable salt thereof.

Embodiment 45. The composition of Embodiment 44, wherein the FXR agonist is obeticholic acid (OCA), GW4064, ASC-42, PX-104, TERN 101, cilofexor, GS-8670, tropifexor, vonafexor, EDP-305, MET-409, or a pharmaceutically acceptable salt thereof.

Embodiment 46. The composition of Embodiment 44, wherein the FXR agonist is:

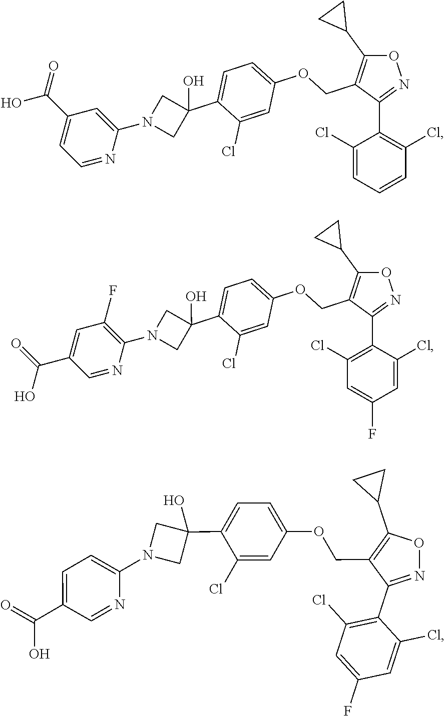

or a pharmaceutically acceptable salt thereof.

Embodiment 47. The composition of any one of Embodiments 44-46, wherein the composition comprises seladelpar lysine.

Embodiment 48. The composition of any one of Embodiments 44-46, wherein the composition comprises seladelpar L-lysine.

Embodiment 49. The composition of any one of Embodiments 44-46, wherein the composition comprises seladelpar L-lysine dihydrate.

Embodiment 50. The composition of any one of Embodiments 44-49, wherein the composition comprises seladelpar or the pharmaceutically acceptable salt thereof which is equivalent to about 5 mg or about 10 mg of seladelpar.

Embodiment 51. A combination of a FXR agonist or a pharmaceutically acceptable salt thereof and seladelpar or a pharmaceutically acceptable salt thereof, for use in a method of treating and/or preventing a liver disease in a patient in need thereof.

Embodiment 52. The combination for use according to Embodiment 51, wherein the FXR agonist is obeticholic acid (OCA), GW4064, ASC-42, PX-104, TERN 101, cilofexor, GS-8670, tropifexor, vonafexor, EDP-305, MET-409, or a pharmaceutically acceptable salt thereof.

Embodiment 53. The combination for use according to Embodiment 51, wherein the FXR agonist is:

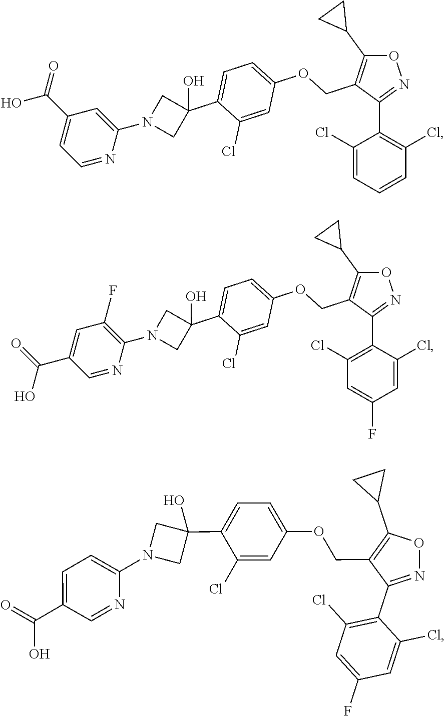

or a pharmaceutically acceptable salt thereof.

Embodiment 54. The combination for use according to any one of Embodiments 51-53, wherein the patient is administered seladelpar lysine.

Embodiment 55. The combination for use according to any one of Embodiments 51-53, wherein the patient is administered seladelpar L-lysine.

Embodiment 56. The combination for use according to any one of Embodiments 1-3, wherein the patient is administered seladelpar L-lysine dihydrate.

Embodiment 57. The combination for use according to any one of Embodiments 51-56, wherein the seladelpar or the pharmaceutically acceptable salt thereof is administered in an amount equivalent to about 10 mg/day of seladelpar.

Embodiment 58. The combination for use according to Embodiments 51-57, wherein the liver disease is metabolic dysfunction-associated steatotic liver disease (MASLD), primary biliary cholangitis (PBC), or primary sclerosing cholangitis (PSC).

Embodiment 59. The combination for use according to Embodiments 51-58, wherein the patient has been previously administered the FXR agonist and the patient was associated with pruritus.

Embodiment 60. The combination for use according to Embodiment 59, wherein the pruritus is FXR-induced.

Embodiment 61. The combination for use according to Embodiment 59 or 60, wherein the pruritus is due to the patient being administered the FXR agonist.

Embodiment 62. The combination for use according to any one of Embodiments 51-61, wherein the FXR agonist and seladelpar are administered together.

Embodiment 63. The combination for use according to any one of Embodiments 51-61, wherein the FXR agonist and seladelpar are administered separately.

Embodiment 64. The combination for use according to any one of Embodiments 51-61, further comprising administering UDCA to the patient.

Embodiment 65. A combination of cilofexor or a pharmaceutically acceptable salt thereof and seladelpar or a pharmaceutically acceptable salt thereof, for use in a method of treating and/or preventing primary biliary cholangitis (PBC) in a patient in need thereof.

Embodiment 66. The combination for use according to Embodiment 65, wherein the patient is administered seladelpar lysine.

Embodiment 67. The combination for use according to Embodiment 65 or 66, wherein the patient is administered seladelpar L-lysine.

Embodiment 68. The combination for use according to any one of Embodiments 65-67, wherein the patient is administered seladelpar L-lysine dihydrate.

Embodiment 69. The combination for use according to any one of Embodiments 65-68, wherein the therapeutically effective amount of seladelpar or the pharmaceutically acceptable salt thereof is equivalent to about 10 mg/day of seladelpar.

Embodiment 70. The combination for use according to any one of Embodiments 65-69, wherein the FXR agonist and seladelpar are administered together.

Embodiment 71. The combination for use according to any one of Embodiments 65-69, wherein the FXR agonist and seladelpar are administered separately.

Embodiment 72. Seladelpar or a pharmaceutically acceptable salt thereof, for use in a method of treating pruritus.

Embodiment 73. Seladelpar or a pharmaceutically acceptable salt thereof for use according to Embodiment 72, wherein the pruritus is Farnesoid X Receptor (FXR)-induced pruritus.

Embodiment 74. Seladelpar or a pharmaceutically acceptable salt thereof for use according to Embodiment 72 or 73, wherein the patient is suffering from a liver disease.

Embodiment 75. Seladelpar or a pharmaceutically acceptable salt thereof for use according to Embodiment 74, wherein the liver disease is selected from: metabolic dysfunction-associated steatohepatitis (MASH), formally non-alcoholic steatohepatitis (NASH), primary sclerosing cholangitis (PSC), primary biliary cirrhosis (PBC), autoimmune hepatitis, alcoholic steatohepatitis (ASH), liver fibrosis, liver cirrhosis, liver steatosis (fatty liver disease), liver ischemia (liver injury in Cortellis), metabolic dysfunction-associated steatotic liver disease (MASLD), and progressive familiar intrahepatic cholestasis (PFIC).

Embodiment 76. Seladelpar or a pharmaceutically acceptable salt thereof for use according to Embodiments 72-74, wherein the patient is suffering from metabolic dysfunction-associated steatotic liver disease (MASLD), primary biliary cholangitis (PBC), or primary sclerosing cholangitis (PSC).

Embodiment 77. Seladelpar or a pharmaceutically acceptable salt thereof for use according to Embodiment 73, wherein the FXR-induced pruritus is due to the patient being administered an FXR agonist or due to a bile acid.

Embodiment 78. Seladelpar or a pharmaceutically acceptable salt thereof for use according to Embodiment 77, wherein the FXR agonist is obeticholic acid (OCA), GW4064, ASC-42, PX-104, TERN 101, cilofexor, GS-8670, tropifexor, vonafexor, EDP-305, MET-409, or a pharmaceutically acceptable salt thereof.

Embodiment 79. Seladelpar or a pharmaceutically acceptable salt thereof for use according to Embodiment 77, wherein the FXR agonist is:

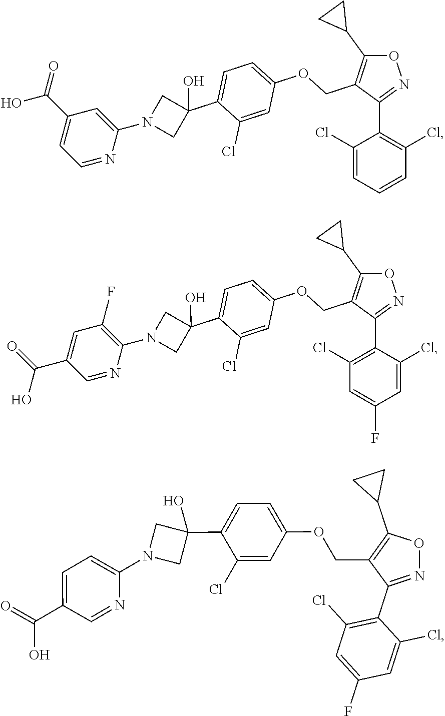

or a pharmaceutically acceptable salt thereof.

Embodiment 80. Seladelpar or a pharmaceutically acceptable salt thereof for use according to Embodiment 73, wherein the FXR-induced pruritus is associated with elevated serum IL-31 levels due to increased serum bile acid levels.

Embodiment 81. A combination of a FXR agonist or a pharmaceutically acceptable salt thereof and seladelpar or a pharmaceutically acceptable salt thereof, for use in a method of improving cholestasis in a patient in need thereof.

Embodiment 82. A combination of a FXR agonist or a pharmaceutically acceptable salt thereof and seladelpar or a pharmaceutically acceptable salt thereof, for use in a method of reducing bile acid in a patient in need thereof.

Embodiment 83. A combination of a FXR agonist or a pharmaceutically acceptable salt thereof and seladelpar or a pharmaceutically acceptable salt thereof, for use in a method of reducing at least one of cholestatic injury, inflammation, or liver fibrosis in a patient in need thereof.

Embodiment 84. The combination for use according to any one of Embodiments 81-83, wherein the FXR agonist is obeticholic acid (OCA), GW4064, ASC-42, PX-104, TERN 101, GS-8670, cilofexor, GS-8670, tropifexor, vonafexor, EDP-305, MET-409, or a pharmaceutically acceptable salt thereof.

Embodiment 85. The combination for use according to any one of Embodiments 81-83, wherein the FXR agonist is:

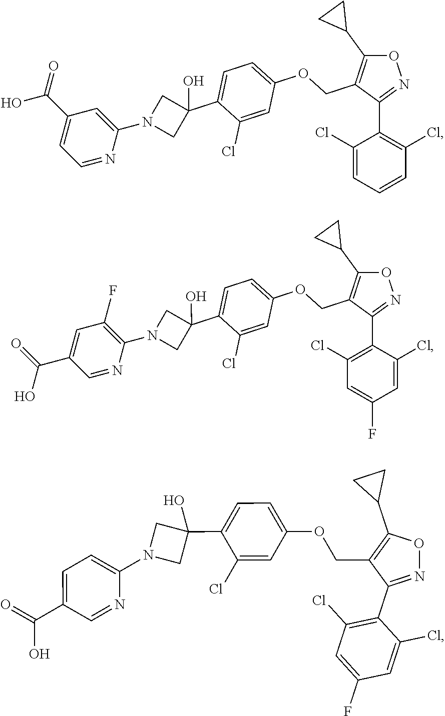

or a pharmaceutically acceptable salt thereof.

Embodiment 86. The combination for use according to any one of Embodiments 81-85, wherein the patient is administered seladelpar lysine.

Embodiment 87. The combination for use according to any one of Embodiments 81-85, wherein the patient is administered seladelpar L-lysine.

Embodiment 88. The combination for use according to any one of Embodiments 81-85, wherein the patient is administered seladelpar L-lysine dihydrate.

Embodiment 89. The combination for use according to any one of Embodiments 81-88, wherein seladelpar or the pharmaceutically acceptable salt thereof is administered in an amount equivalent to about 10 mg/day of seladelpar.

Embodiment 90. The combination for use according to any one of Embodiments 81-89, wherein the patient is suffering from a liver disease.

Embodiment 91. The combination for use according to any one of Embodiments 90, wherein the patient is suffering from metabolic dysfunction-associated steatotic liver disease (MASLD), primary biliary cholangitis (PBC), or primary sclerosing cholangitis (PSC).

Embodiment 92. The combination for use according to any one of Embodiments 81-91, wherein the FXR agonist and seladelpar are administered together.

Embodiment 93. The combination for use according to any one of Embodiments 81-91, wherein the FXR agonist and seladelpar are administered separately.

Embodiment 94. A composition comprising a therapeutically effective amount of an FXR agonist and a therapeutically effective amount of seladelpar or a pharmaceutically acceptable salt thereof, for use in a method of treating or preventing a liver disease or pruritus in a patient in need thereof.

Embodiment 95. The composition for use according to Embodiment 94, wherein the FXR agonist is obeticholic acid (OCA), GW4064, ASC-42, PX-104, TERN 101, cilofexor, GS-8670, tropifexor, vonafexor, EDP-305, MET-409, or a pharmaceutically acceptable salt thereof.

Embodiment 96. The composition for use according to Embodiment 94, wherein the FXR agonist is:

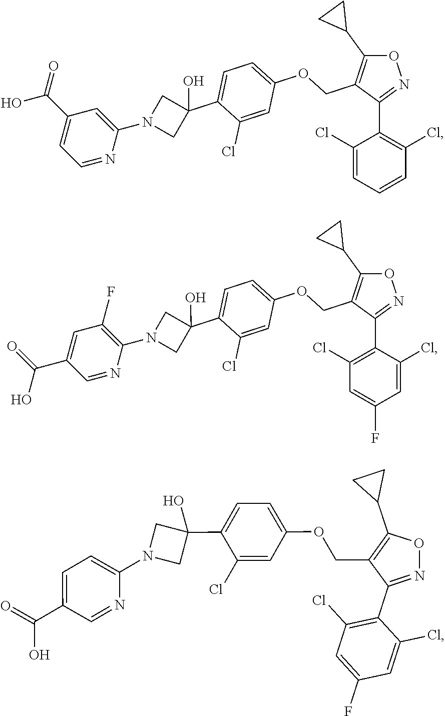

or a pharmaceutically acceptable salt thereof.

Embodiment 97. The composition for use according to any one of Embodiments 94-96, wherein the composition comprises seladelpar lysine.

Embodiment 98. The composition for use according to any one of Embodiments 94-96, wherein the composition comprises seladelpar L-lysine.

Embodiment 99. The composition for use according to any one of Embodiments 94-96, wherein the composition comprises seladelpar L-lysine dihydrate.

Embodiment 100. The composition for use according to any one of Embodiments 94-99, wherein the composition comprises seladelpar or the pharmaceutically acceptable salt thereof which is equivalent to about 5 mg or about 10 mg of seladelpar.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Serum Bile Acids

Fifteen bile acids in fasting serum samples from PBC patients in Phase 3 Study and matched healthy volunteers were analyzed to assess whether any individual bile acids were associated with pruritus. Liquid chromatography tandem mass spectrometry was used to measure the following bile acids: unconjugated bile acids (cholic acid (CA), chenodeoxycholic acid (CDCA), deoxycholic acid (DCA), lithocholic acid (LCA) and ursodeoxycholic acid (UDCA)), glycine-conjugated bile acids (glycocholic acid (GCA), glycochenodeoxycholic acid (GCDCA), glycodeoxycholic acid (GDCA), glucolithocholic acid (GLCA) and glycoursodeoxycholic acid (GUDCA), and taurine-conjugated bile acids (taurocholic acid (TCA), taurochenodeoxycholic acid (TCDCA), taurodeoxycholic acid (TDCA), taurolithocholic acid (TLCA) and tauroursodeoxycholicacid (TUDCA)).

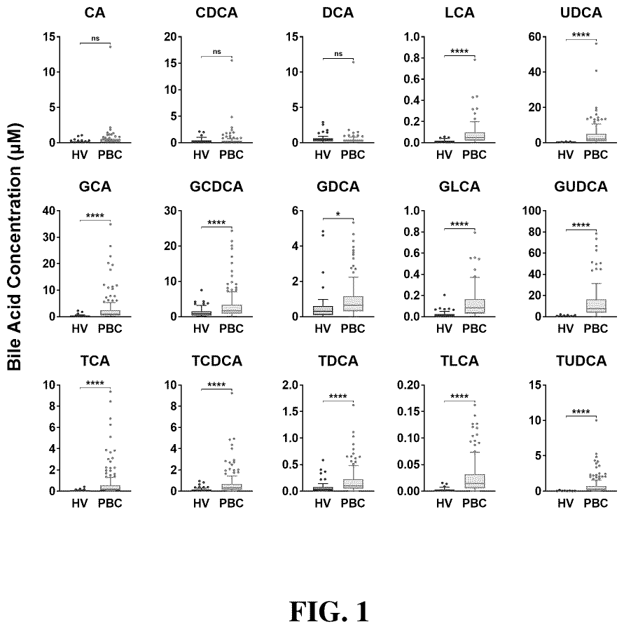

Levels of individual serum bile acids in patients with PBC compared to healthy volunteers (“HV”) is shown in FIG. 1. As shown in FIG. 1, compared to healthy volunteers, individual serum bile acids were substantially elevated in patients with PBC. Unconjugated bile acids, except for LCA, which was present at very low levels, were not different between the two groups. In contrast, glycine- and taurine-conjugated primary bile acids (GCA, GCDCA, TCA and TCDCA) were substantially elevated in PBC patients compared to healthy volunteers.

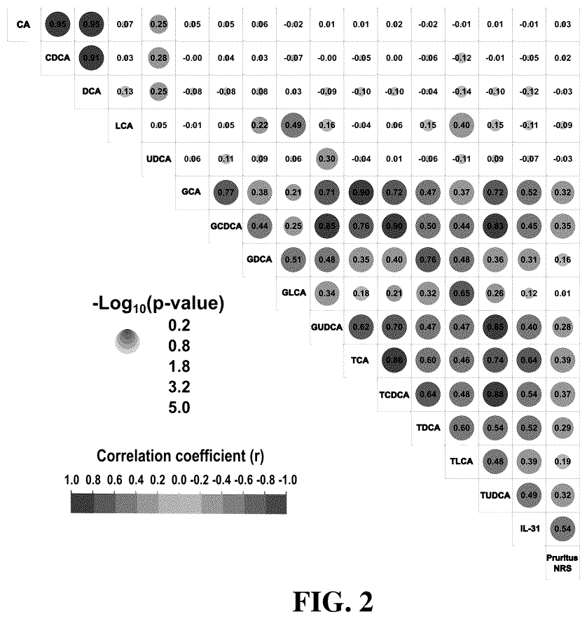

Correlations between serum conjugated bile acids in patients with PBC are shown in FIG. 2. In FIG. 2, size of circle reflects statistical significance (−Log 10(p-value)), color of circle indicates correlation (+1 (red) to −1 (blue)) and the numeral indicates the correlation coefficient (r). As shown in FIG. 2, serum levels of individual conjugated bile acids were highly correlated with each other. The high levels of UDCA and its conjugated forms observed in PBC patients were due to its use as a treatment in PBC patients. Interestingly GUDCA and TUDCA showed strong correlations with conjugated primary bile acids GCA (r=0.71, r=0.72), GCDCA (r=0.85, r=0.83), TCA (r=0.62, r=0.74) and TCDCA (r=0.70, r=0.88) while UDCA did not reveal any correlation.

### Example 2: Regulation of IL-31 Production by FXR Agonists and Seladelpar in Primary Human Hepatocytes

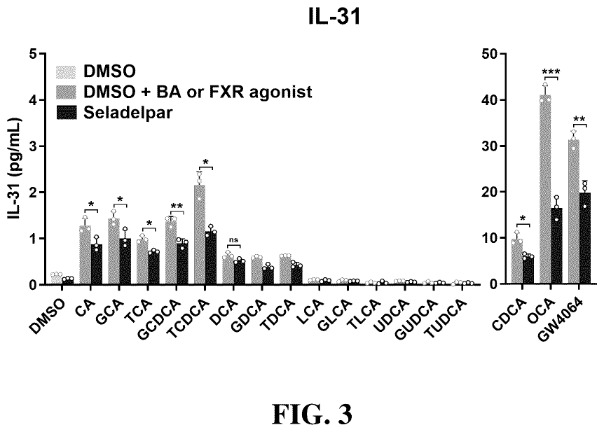

Given that each conjugated bile acid rose in concert with one another, we tested individual bile acids along with synthetic FXR agonists for their capacity to induce IL-31 in primary human hepatocytes. Human hepatocytes were cultured and treated with were treated for 72 hours with bile acids, OCA, or GW4064, either with or without seladelpar (0.5 μM) or DY268. After treatment, the cell culture medium was concentrated and human IL-31 was quantified in the concentrated supernatants. The IL-31 levels are shown in FIG. 3.

As shown in FIG. 3, bile acids and FXR agonists significantly increased the levels of IL-31. The levels of IL-31 protein were 41 μg/mL for OCA, 31 μg/mL for GW4064, and 10 μg/mL for CDCA, compared to just 0.23 pg/mL in DMSO-treated hepatocytes. Additionally, other bile acids—CA (1.28 pg/mL), GCA (1.43 pg/mL), TCA (0.99 pg/mL), GCDCA (1.37 pg/mL), TCDCA (2.17pg/mL), DCA (0.64 pg/mL), GDCA (0.60 pg/mL), and TDCA (0.63 pg/mL)—also increased IL-31 protein levels. However, treatment with LCA, UDCA, and their conjugates resulted in IL-31 levels lower than those of DMSO-treated cells.

On the other hand, seladelpar (0.5 μM) cotreatment significantly reduced IL-31 protein levels in the media of hepatocytes treated with FXR agonists OCA (−60%, p<0.001) and GW4064 (−37%, p<0.01), as well as with bile acids CDCA (−39%, p<0.05) and TCDCA (−47%, p<0.05).

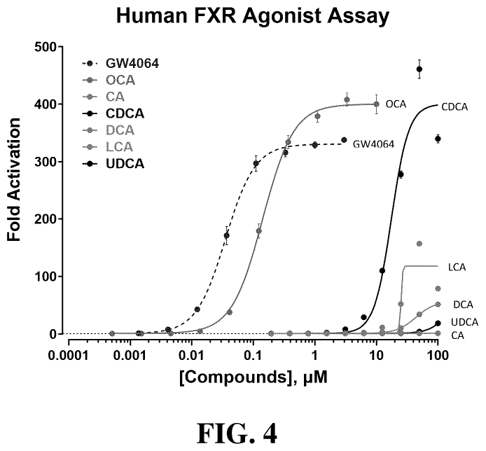

Next, to assess the agonist activities of bile acids on human FXR, bile acids were tested in human FXR reporter CHO cells expressing hybrid receptors in which the N-terminal DNA binding domain (DBD) of human FXR was replaced with the yeast Ga14 DBD and the Ga14 upstream activating sequence functionally linked to firefly luciferase. Human FXR reporter cell suspensions were incubated with either OCA, GW4064, or bile acids at 37° C. for 22 hours. Each treatment was performed in triplicate. After incubation, the media were removed, luciferase detection reagent was added, and luminescence was measured after a 10-minute incubation period at room temperature. Dose-response curves for OCA, GW4064, and bile acids were generated by non-linear curve-fitting of fold-activation versus Log₁₀[Compound] using GraphPad Prism software, which is shown in FIG. 4.

As shown in FIG. 4, the production of IL-31 in primary human hepatocytes aligned with the ability of these agents to activate FXR. For example, GW4064 and OCA were the most potent FXR agonists, with EC₅₀ values of 0.04 μM and 0.14 μM, respectively. CDCA acted as a full agonist with an EC₅₀ of 17.6 μM. However, the primary bile acid CA did not cause FXR activation, nor did conjugated bile acids. CHO cells do not have transporters for conjugated bile acids, so they would not display activity in this human FXR reporter gene assay. CDCA induced more IL-31 production than GCDCA and TCDCA.

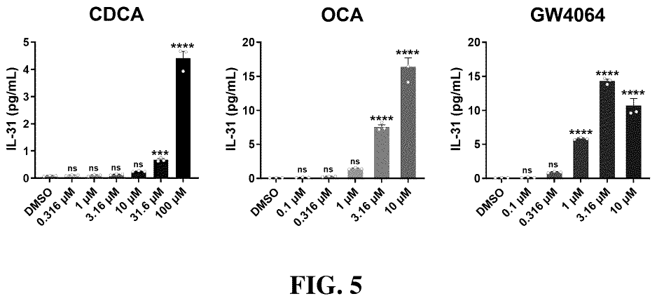

Next, whether IL-31 protein production varied with concentration of FXR agonist was analyzed. Treatment of primary human hepatocytes with CDCA, OCA, or GW4064 resulted in significant, concentration-dependent increases in IL-31 protein levels in the media, as shown in FIG. 5. Consistent with the observed IL-31 increases by FXR activation, the FXR antagonist DY268 blocked CDCA-induced IL-31 production (−92%, p<0.01) (FIG. 3B).

In view of above data, it was found that CDCA, CA and their conjugates stimulated IL-31 production. CDCA, a full FXR agonist, along with synthetic full FXR agonists OCA and GW4064, effectively induced IL-31 production in a concentration-dependent manner reflecting the relative potencies of these agonists. Notably, UDCA, which is the first line therapy for PBC, is not an FXR agonist and is not demonstrated to cause pruritus, did not increase IL-31 production in primary human hepatocytes.

On the other hand, consistent with the clinical observations, seladelpar significantly reduced IL-31 production stimulated by bile acids in primary human hepatocytes. Seladelpar also attenuated synthetic FXR agonist-induced IL-31 production. Thus, it is contemplated that seladelpar can act at the level of hepatocytes to attenuate IL-31 production.

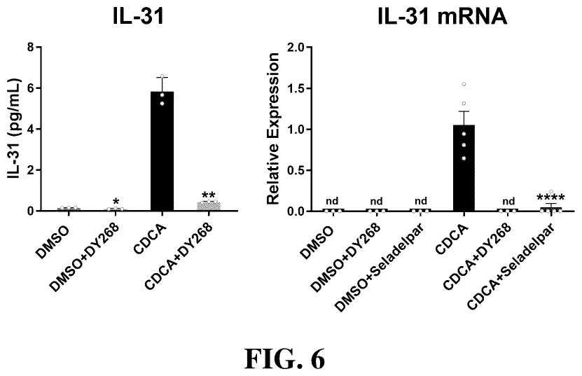

To evaluate IL-31 expression, primary human hepatocytes were treated with CDCA (100 μM), OCA (10 μM), GW4064 (3 μM), DY268 (10 μM) or seladelpar (0.5 μM) for 72 hours. RNA was then extracted. The concentration and purity of the isolated RNA were measured with a NanoDrop spectrophotometer. For reverse transcription, 400 ng of RNA was converted to cDNA using the Quantitect Reverse Transcription Kit (Qiagen, Cat #205311). IL-31 mRNA expression was quantified using qPCR for each sample using PrimeTime™ Gene Expression Master Mix (Integrated DNA Technologies (IDT), Cat #1055771). PrimeTime™ qPCR primer assays (Integrated DNA Technologies) were used for RT-qPCR analysis of IL-31. Gene expression levels were calculated using the AACt method, with HPRT1 serving as the housekeeping gene. Effect of FXR antagonist DY268 (10 μM) on IL-31 protein in primary human hepatocytes treated with CDCA (100 μM), and effect of FXR antagonist DY268 (10 μM) or seladelpar (0.5 μM) on IL-31 mRNA levels in primary human hepatocytes treated with CDCA (100 μM) using RT-qPCR with expression relative to CDCA are shown in FIG. 6.

As shown in FIG. 6, IL-31 mRNA levels were not detectable by RT-qPCR in the absence of CDCA treatment in hepatocytes. Corresponding with IL-31 protein levels, CDCA resulted in measurable levels of IL-31 mRNA, and FXR antagonist DY268 resulted in a return to undetectable levels of IL-31 mRNA. Seladelpar treatment reduced IL-31 protein levels induced either by bile acids or FXR agonists as shown in FIG. 3, and its effects were consistent with IL-31 mRNA levels shown in FIG. 6. This provides evidence that IL-31 production is mediated by FXR activation of transcription and that seladelpar attenuates FXR-dependent transcriptional activation of IL-31.

### Example 3: Identification of FXR Binding Sites in the IL-31 Gene

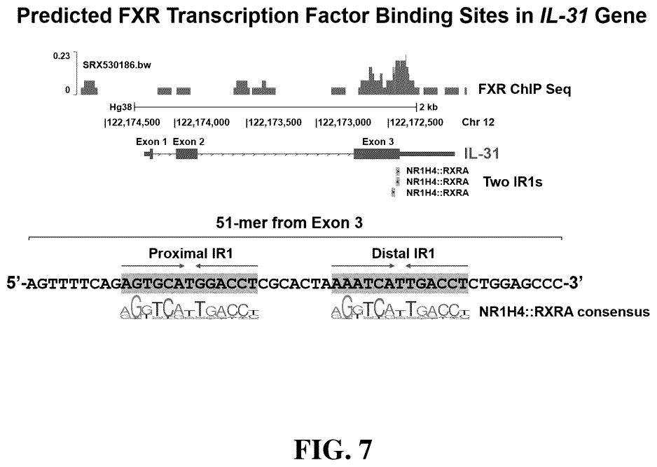

It was investigated whether the increase in IL-31 production induced by FXR agonists in primary human hepatocytes is due to direct transcriptional activation of the human IL-31 gene, which was hypothesized to occur through the interaction of FXR and RXRA heterodimers (NR1H4::RXRA) with IR1 sequences within the IL-31 gene. Using the JASPAR database of predicted transcription factor binding sites, sites on the UCSC Genome Browser were reviewed and two adjacent predicted FXR binding sites (IR1s) were identified. These sites are separated by 7 base pairs, located in exon 3 of the IL-31 gene and are less than 2 kb from the transcription start site (TSS). Additionally, ChIP-Seq data from GW4064-treated hepatocytes (GME: SRX530186.bw) were overlaid on the IL-31 gene in the Integrative Genomics Viewer, revealing that the highest peaks of FXR-DNA interaction within this gene are centered in the same region as shown in FIG. 7, which is alignment of the genomic sequence of the human IL-31 gene with FXR ChIP-Seq data (GME: SRX530186.bw) and JASPAR-predicted FXR:RXRA binding sites.

Then, a pentamer of the 51-mer region containing both IR1sequence elements was evaluated for its ability to activate luciferase reporter gene transcription in response to FXR agonists when transfected into Huh7 cells, which endogenously express the FXR protein. Huh7 cells in a 96-well plate were transfected with pRMT-luc luciferase reporter plasmid constructs (Origene), which contained five tandem head-to-tail copies of the 51-mer sequence, a minimal promoter (MinP) containing a TATA box, and a firefly luciferase reporter using Lipofectamine 3000 reagent. The following day, the cells were treated with DMSO, CDCA, OCA, or GW4064 for 24 hours, and luciferase activity was measured using the Bright-Glo™ luciferase assay system (Promega). To determine which of the two IR1 sites (proximal or distal) was more critical for transcriptional activity, we also tested mutated versions of the plasmid construct. These mutant versions of the 51-mer sequence were compared to the wild-type 51-mer to evaluate their responses to the FXR agonists CDCA, OCA and GW4064 in three independent experiments.

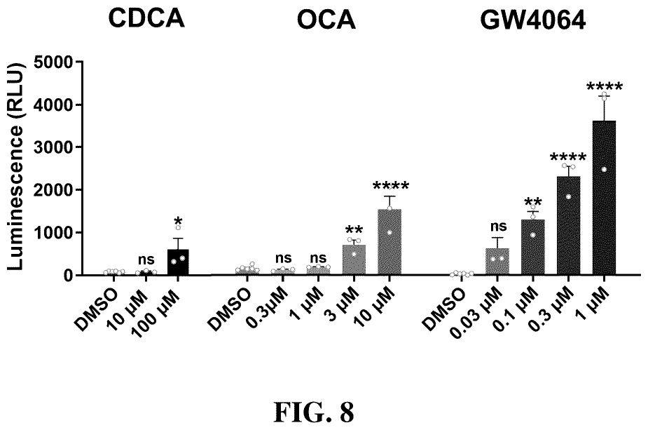

The luciferase activity is shown in FIG. 8. As shown in FIG. 8, cells treated with increasing concentrations of CDCA, OCA, and GW4064 showed elevated luciferase activity. The IR1 elements provided concentration-dependent transcriptional responsiveness to these compounds, reflecting their potency as FXR agonists. Thus, IL-31 Exon 3 IR1 sites can mediate concentration-dependent reporter gene activity driven by FXR agonists in Huh7 cells.

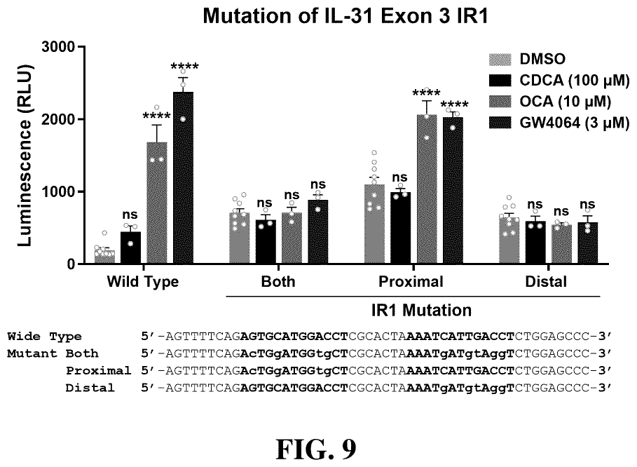

On the other hand, mutant versions of the IL-31 Exon 3 IR1 sites showed differential abilities to support FXR agonist-driven reporter gene transcription, as shown in FIG. 9. Cells transfected with a plasmid containing mutant versions of both IR1 sequence elements (proximal and distal to TSS) that diverged from the IR1 consensus were not induced by FXR agonists, indicating that the IR1 elements are essential for this response. Further studies with plasmids containing mutations in the proximal or distal IR1 sequences demonstrated varying effects on FXR agonist-driven transcription. Mutation of the IR1 sites led to increased basal luciferase activity, which suggests that unliganded FXR may repress transcription in this context. Both IR1 elements appear to be required for full activity; however, mutation of the distal IR1 completely eliminated luciferase activity induced by FXR agonists. In contrast, the construct with the mutant proximal IR1 retained some activity.

### Example 4: Cut & Run qPCR Demonstrates Interaction of FXIt Protein with IR1 Sites in the IL-31 Gene in Primary Human Hepatocytes

To evaluate whether FXR interacts with the IR1 sites in Exon 3 of the IL-31 gene in live primary human hepatocytes treated with GW4064, we performed qPCR amplification using a single pair of primers that covers both IR1 sites.

Approximately 1.5×10⁶ primary human hepatocytes were treated with 3 μM GW4064 for 72 hours and then harvested. The cells were processed immediately with the CUT&RUN (Cleavage Under Targets & Release Using Nuclease) Assay Kit (Cell Signaling Technology, Cat #86652). For binding and the subsequent cleavage and release steps, FXR/NR1H4 (E4B8P) mouse monoclonal antibody (Cell Signaling Technology, Cat #72105S) and a negative control mouse monoclonal antibody IgG isotype (E5Y6Q) (Cell Signaling Technology, Cat #61656) were used at a concentration of 6 μg/mL. DNA was then purified using DNA purification buffers and spin columns. Finally, binding of FXR to the IR1 FXR:RXRA sites of the IL-31 gene, as well as to the positive control gene SLC10A1 (BSEP) was quantified by qPCR. Ct values were normalized to IgG controls in three replicates. The chromosomal positions of the PCR primers with reference to human genome assembly hg38, primer sequences for qPCR analysis, the amplicon size and sequences are as follows:

IL-31 (chr12:122172437+122172496)
(SEQ ID NO: 1)
Forward, 5′-GAGGTCAATGATTTTAGTGCGAGG-3′;
(SEQ ID NO: 2)
Reverse, 5′-CCTGACTATTTCTCAACAGTTTTCA-3′;
60 bp,
(SEQ ID NO: 3)
GAGGTCAATGATTTTAGTGCGAGGtccatgcactcTGAAAACTGTTGAG
AAATAGTCAGG
BSEP (chr2:169031365+169031424)
(SEQ ID NO: 4)
Forward, 5′-CTATTTGCCTAAGGATCAATGTCCC-3′;
(SEQ ID NO: 5)
Reverse, 5′-TCGTATGTCACTGAACTGTGCT-3′;
60 bp,
(SEQ ID NO: 6)
CTATTTGCCTAAGGATCAATGTCCCtaagggcagcccaAGCACAGTTCA
GTGACATACGA.

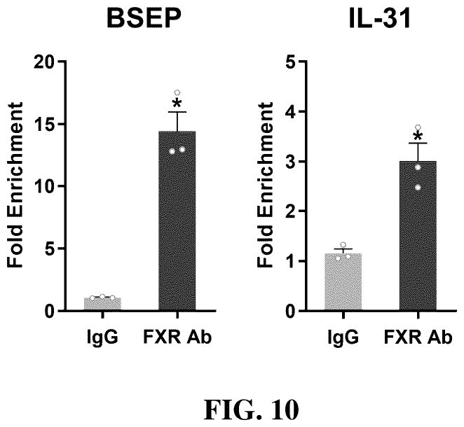

As shown in FIG. 10, FXR-dependent pull-down of this BSEP IR1 FXR site resulted in 14-fold enrichment of the qPCR signal for BSEP relative to background isotypic IgG. The qPCR signal from the 60-nucleotide region encompassing both IL-31 exon 3 IR1 sites was 3-fold above background IgG. This demonstrates the direct interaction between FXR and the IL-31 gene in living hepatocytes.

Thus, it is confirmed that FXR binds to the chromatin region containing the IR1 sites in exon 3. Together, these in vitro studies suggest that FXR activates IL-31 transcription through the IR1 sites in the presence of CDCA or synthetic FXR agonists, leading to increased IL-31 mRNA levels and subsequent release of IL-31 protein from hepatocytes, while seladelpar in opposite can reduce IL-31 expression in hepatocytes even in the presence of excess amount of FXR agonists.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation, or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement, and variation of the inventions embodied therein herein disclosed may be resorted to by those skilled in the art, and that such modifications, improvements, and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A method of treating and/or preventing a liver disease in a patient in need thereof, the method comprising administering a therapeutically effective amount of a FXR agonist or a pharmaceutically acceptable salt thereof and a therapeutically effective amount of seladelpar or a pharmaceutically acceptable salt thereof to the patient.

2. The method of claim 1, wherein the FXR agonist is obeticholic acid (OCA), GW4064, ASC-42, PX-104, TERN 101, cilofexor, GS-8670, tropifexor, vonafexor, EDP-305, MET-409, or a pharmaceutically acceptable salt thereof.

3. The method of claim 1, wherein the FXR agonist is: 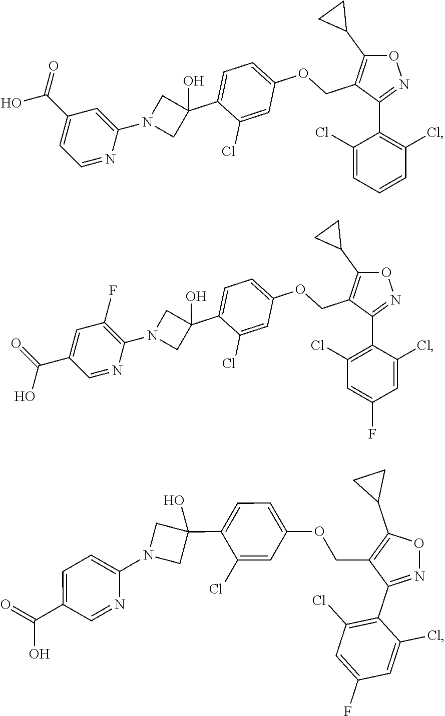
or a pharmaceutically acceptable salt thereof.

4. The method of claim 1, wherein the patient is administered seladelpar lysine.

5. The method of claim 1, wherein the patient is administered seladelpar L-lysine.

6. The method of claim 1, wherein the patient is administered seladelpar L-lysine dihydrate.

7. The method of claim 1, wherein the therapeutically effective amount of seladelpar or the pharmaceutically acceptable salt thereof is equivalent to about 10 mg/day of seladelpar.

8. The method of claim 1, wherein the liver disease is metabolic dysfunction-associated steatotic liver disease (MASLD), primary biliary cholangitis (PBC), or primary sclerosing cholangitis (PSC).

9-11. (canceled)

12. The method of claim 1, wherein the FXR agonist and seladelpar are administered together.

13. The method of claim 1, wherein the FXR agonist and seladelpar are administered separately.

14. The method of claim 1, further comprising administering UDCA to the patient.

15. A method of treating and/or preventing primary biliary cholangitis (PBC) in a patient in need thereof, the method comprising administering a therapeutically effective amount of cilofexor or a pharmaceutically acceptable salt thereof and a therapeutically effective amount of seladelpar or a pharmaceutically acceptable salt thereof to the patient.

16. The method claim 15, wherein the patient is administered seladelpar lysine.

17-18. (canceled)

19. The method of claim 15, wherein the therapeutically effective amount of seladelpar or the pharmaceutically acceptable salt thereof is equivalent to about 10 mg/day of seladelpar.

20. The method of claim 15, wherein the FXR agonist and seladelpar are administered together.

21. The method of claim 15, wherein the FXR agonist and seladelpar are administered separately.

22-30. (canceled)

31. A method of improving cholestasis in a patient in need thereof, the method comprising administering a therapeutically effective amount of a FXR agonist or a pharmaceutically acceptable salt thereof and a therapeutically effective amount of seladelpar or a pharmaceutically acceptable salt thereof to the patient.

32. A method of reducing bile acid in a patient in need thereof, the method comprising administering a therapeutically effective amount of a FXR agonist or a pharmaceutically acceptable salt thereof and a therapeutically effective amount of seladelpar or a pharmaceutically acceptable salt thereof to the patient.

33. A method of reducing at least one of cholestatic injury, inflammation, or liver fibrosis in a patient in need thereof, the method comprising administering a therapeutically effective amount of a FXR agonist or a pharmaceutically acceptable salt thereof and a therapeutically effective amount of seladelpar or a pharmaceutically acceptable salt thereof to the patient.

34-43. (canceled)

44. A composition comprising a therapeutically effective amount of an FXR agonist and a therapeutically effective amount of seladelpar or a pharmaceutically acceptable salt thereof.

45-49. (canceled)


SEQUENCE LISTING (6 sequences)

SEQ ID NO: 1 - DNA, 24 bp, synthetic construct
  source 1..24: mol_type = other DNA
gaggtcaatgattttagtgcgagg

SEQ ID NO: 2 - DNA, 25 bp, synthetic construct
  source 1..25: mol_type = other DNA
cctgactatttctcaacagttttca

SEQ ID NO: 3 - DNA, 60 bp, synthetic construct
  source 1..60: mol_type = other DNA
gaggtcaatgattttagtgcgaggtccatgcactctgaaaactgttgagaaatagtcagg

SEQ ID NO: 4 - DNA, 25 bp, synthetic construct
  source 1..25: mol_type = other DNA
ctatttgcctaaggatcaatgtccc

SEQ ID NO: 5 - DNA, 22 bp, synthetic construct
  source 1..22: mol_type = other DNA
tcgtatgtcactgaactgtgct

SEQ ID NO: 6 - DNA, 60 bp, synthetic construct
  source 1..60: mol_type = other DNA
ctatttgcctaaggatcaatgtccctaagggcagcccaagcacagttcagtgacatacga